SEQUENCE LISTING (30 sequences)

SEQ ID NO: 1 - protein, 120 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARGGGSWYSNWFDPWGQGTMVTVSS

SEQ ID NO: 2 - protein, 107 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQDRSLPPTFGGGTKVEIK

SEQ ID NO: 3 - protein, 121 aa, synthetic construct
QVQLQQWGAGLLKPSETLSLTCAVYGGSFSGIHWNWIRQPPGKGLEWIGDIDTSGSTNYNPSLKSRVTISVDTSKNQFSLKLSSVTAADTAVYYCARLGQESATYLGMDVWGQGTTVTVSS

SEQ ID NO: 4 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQLYTYPFTFGGGTKVEIK

SEQ ID NO: 5 - protein, 124 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSS

SEQ ID NO: 6 - protein, 107 aa, synthetic construct
DIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIK

SEQ ID NO: 7 - protein, 125 aa, synthetic construct
QVQLVQSGAEVKKPGASVKVSCKASGYTFKEYGISWVRQAPGQGLEWMGWISAYSGHTYYAQKLQGRVTMTTDTSTSTAYMELRSLRSDDTAVYYCARGPHYDDWSGFIIWFDPWGQGTLVTVSS

SEQ ID NO: 8 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYRFPPTFGQGTKVEIK

SEQ ID NO: 9 - protein, 124 aa, synthetic construct
QVQLQESGPGLVKPSETLSLTCTVSGGSISSPDHYWGWIRQPPGKGLEWIGSIYASGSTFYNPSLKSRVTISVDTSKNQFSLKLSSVTAADTAVYYCARETDYSSGMGYGMDVWGQGTTVTVSS

SEQ ID NO: 10 - protein, 107 aa, synthetic construct; identical to SEQ ID NO: 6

SEQ ID NO: 11 - protein, 123 aa, synthetic construct
QITLKESGPTLVKPTQTLTLTCTFSGFSLDTEGVGVGWIRQPPGKALEWLALIYFNDQKRYSPSLKSRLTITKDTSKNQVVLTMTNMDPVDTAVYYCARDTGYSRWYYGMDVWGQGTTVTVSS

SEQ ID NO: 12 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQAYAYPITFGGGTKVEIK

SEQ ID NO: 13 - protein, 127 aa, synthetic construct
QVQLQQWGAGLLKPSETLSLTCAVYGGSFEKYYWSWIRQPPGKGLEWIGEIYHSGLTNYNPSLKSRVTISVDTSKNQFSLKLSSVTAADTAVYYCARVRYDSSDSYYYSYDYGMDVWGQGTTVTVSS

SEQ ID NO: 14 - protein, 113 aa, synthetic construct
DIVLTQSPDSLAVSLGERATINCKSSQSVLYSSNNKNYLAWYQQKPGQPPKLLIYWASSRESGVPDRFSGSGSGTDFTLTISSLQAEDVAVYYCQQSYSFPWTFGGGTKVEIK

SEQ ID NO: 15 - protein, 127 aa, synthetic construct
QVQLQQWGAGLLKPSETLSLTCAVYGGSFSRYVWSWIRQPPGKGLEWIGEIDSSGKTNYNPSLKSRVTISVDTSKNQFSLKLSSVTAADTAVYYCARVRYDSSDSYYYSYDYGMDVWGQGTTVTVSS

SEQ ID NO: 16 - protein, 113 aa, synthetic construct; identical to SEQ ID NO: 14

SEQ ID NO: 17 - protein, 127 aa, synthetic construct
QVQLQQWGAGLLKPSETLSLTCAVYGGSFSGYAWSWIRQPPGKGLEWIGEIDHRGFTNYNPSLKSRVTISVDTSKNQFSLKLSSVTAADTAVYYCARVRYDSSDSYYYSYDYGMDVWGQGTTVTVSS

SEQ ID NO: 18 - protein, 113 aa, synthetic construct; identical to SEQ ID NO: 14

SEQ ID NO: 19 - protein, 123 aa, synthetic construct
QVQLQQWGAGLLKPSETLSLTCAVYGGSFQKYYWSWIRQPPGKGLEWIGEIDTSGFTNYNPSLKSRVTISVDTSKNQFSLKLSSVTAADTAVYYCARVGRYSYGYYITAFDIWGQGTTVTVSS

SEQ ID NO: 20 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQHYSFPFTFGGGTKVEIK

SEQ ID NO: 21 - protein, 120 aa, synthetic construct
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 22 - protein, 107 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 23 - protein, 120 aa, synthetic construct
EVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 24 - protein, 107 aa, synthetic construct
DIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIK

SEQ ID NO: 25 - protein, 245 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 26 - protein, 1018 aa, synthetic construct
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRSGSGEGRGSLLTCGDVEENPGPMALPVTALLLPLALLLHAARPQLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSSGSTSGSGKPGSGEGSTKGDIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIKAAAFVPVFLPAKPTTTPAPRPPTPAPTIASQPLSLRPEACRPAAGGAVHTRGLDFACDIYIWAPLAGTCGVLLLSLVITLYCNHRNRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 733 aa, synthetic construct
MLLLVTSLLLCELPHPAFLLIPDIQMTQSPSSLSASVGDRVTITCRASQSINSYLNWYQQKPGKAPKLLIYAASSLQSGVPSRFSGSGSGTDFTLTISSLQPEDFATYYCQQSLADPFTFGGGTKVEIKGGGGSGKPGSGEGGSQLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARETDYSSGMGYGMDVWGQGTTVTVSSGGGGSGKPGSDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGGGGSGGGGSGGGGSEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 32 bp, synthetic construct
  source 1..32: mol_type = other DNA
cagttgaagtcggaagtttacatacacytaag

SEQ ID NO: 29 - DNA, 32 bp, synthetic construct
  source 1..32: mol_type = other DNA
yccagtgggtcagaagtttacatacactmart

SEQ ID NO: 30 - protein, 340 aa, synthetic construct
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

(19) United States
(12) Patent Application Publication
(10) Pub. No.: US 2024/0000844 A1
(43) Pub. Date: Jan. 4, 2024

(54) NON-VIRAL DELIVERY OF CELL THERAPY CONSTRUCTS

(71) Applicant: Kite Pharma, Inc., Foster City, CA (US)

(72) Inventors: Qi Cai, Champaign, IL (US); Hsing-Chuan Tsai, Santa Monica, CA (US); Kaiyuan Jiang, King Of Prussia, PA (US)

(21) Appl. No.: 18/323,914

(22) Filed: May 25, 2023

Related U.S. Application Data
(60) Provisional application No. 63/492,110, filed on Mar. 24, 2023.
(60) Provisional application No. 63/346,547, filed on May 27, 2022.

(51) Int. Cl. A61K 35/17; A61K 39/00

(52) U.S. Cl. CPC A61K 35/17; A61K 39/4611; A61K 39/4631; A61K 2239/28; A61K 2239/30; A61K 2239/22

(57) ABSTRACT

The present disclosure provides transposon-based systems for introducing cellular therapeutic products, such as CAR and TCR, into a target immune cell. The transposon-based systems can carry larger payloads than conventional viral vector-based technologies, simplifying multi-genetic editing and can reduce undesired recombination between homologous sequences in the payload. Also provided is a shortened autologous process that can be completed within a few days, within one day or even within a few hours. Even without immune cell activation, enrichment or expansion, the resulting cell populations achieve greatly higher in vivo therapeutic efficacy than the much lengthier autologous process that employs viral vectors.

### CROSS REFERENCE TO RELATED APPLICATIONS

This application claims the benefit of U.S. Provisional Patent Application No. 63/346,547, filed on May 27, 2022 and U.S. Provisional Patent Application No. 63/492,110 filed Mar. 24, 2023, both of which are hereby incorporated by reference their entireties.

### SEQUENCE LISTING

The instant application contains a Sequence Listing which has been submitted electronically in XML file format and is hereby incorporated by reference in its entirety. Said XML copy, created on Aug. 25, 2023, is named K-1139-US-NP_SL.xml and is 31,958 bytes in size.

### BACKGROUND

Cell therapies employ enriched or modified human immune cells to target and kill cancer cells in a patient. To increase the ability of immune cells to target and kill a particular cancer cell, methods have been developed to engineer immune cells to express constructs which direct the immune cells to a particular target cancer cell. Chimeric antigen receptors (CARs) and engineered T cell receptors (TCRs), which include binding domains capable of interacting with a particular tumor antigen, allow the immune cells to target and kill cancer cells that express the particular tumor antigen.

A major challenge in the preparation of CAR or TCR cells is the transfer of the coding sequences to a target immune cell. Currently, the vast majority of CAR T-cell production relies on the transfer by viral vectors. Retroviral genes in combination with inducible promoters can enhance transduction rates and produce relatively large numbers of CAR-containing T-cells.

Vector-based murine leukemia virus (MLV) is the most commonly used gamma retroviral vector. The use of MLV, however, has been associated with T-cell-related leukemia. Vectors derived from another retroviral family, Lentivirus, have shown better integration efficiency. Lentiviral vectors are able to target nondividing cells, which has been a major challenge for MLV-based vectors.

Viral vectors are commonly used in CAR T-cell production, but significant challenges remain. For instance, the viral vectors are inherently associated with oncogenic and mutagenic potentials. Moreover, the use of viruses in current Good Manufacturing Practice (cGMP) laboratories is burdened with strict regulations. Also important, lentiviral/retroviral transduction is limited by the size of the viral capsid, therefore setting a limit on the size of transgene. Further, the size of the transgene can be limited by the effects of the gene of interest and/or multiple gene expression on the physiology of the viral vector itself, including potential gene toxicity to the virus caused by the transgene.

Yet another significant challenge comes with the highly complicated autologous cell engineering and production process, which typically takes at least a week, and can take as long as several weeks. During the process, lymphocytes collected from the patients must be shipped to the process center, while the produced cells have to be cryopreserved and then shipped back to the patient for implantation. This highly complicated process necessarily leads to high costs, and limited clinical applications.

### SUMMARY

The present disclosure, in various embodiments, provides transposon-based systems for introducing cellular therapeutic products, such as CAR and TCR, into a target immune cell. The transposon-based systems can carry larger payloads than conventional viral vector-based technologies and can reduce undesired recombination between homologous sequences in the payload. In certain aspects, transposon-based systems simplify multi-genetic editing to one step transfection. Also provided is a shortened autologous process that can be completed within a few days or even within the same day. In certain aspects, the shortened autologous process can be completed within 4 hours. Even without immune cell activation, enrichment or expansion, the resulting cell populations achieve greatly higher in vivo therapeutic efficacy than the much lengthier autologous process that employs viral vectors.

This shortened process can be easily incorporated into an enclosed automated device and carried out at a preferred location (for example, a clinical site) other than at centralized manufacturing sites. This has not been practical for the conventional autologous technology. The two ways shipping required for the conventional technology can be skipped. Also importantly, the freeze/thaw steps needed for transportation can also be eliminated, which will further improve both the patient care and the quality of the product.

In accordance with one embodiment of the present disclosure, provided is a transposon comprising a transgene encoding a polypeptide that comprises a first chimeric antigen receptor (CAR) and a second CAR, wherein the first CAR and the second CAR each comprises a single chain fragment (scFv), a transmembrane domain, and an immunoreceptor tyrosine-based activation motif (ITAM).

In some embodiments, the transposon is a DNA transposon selected from the group consisting of a Sleeping Beauty transposon, a piggyBac transposon, and a Tc Buster transposon, or a retro-transposon such as R2 transposon. In some embodiments, the transposon is a Sleeping Beauty transposon.

In some embodiments, the transgene is at least 5000 nucleotides in length. In some embodiments, the transgene is at least 6000, 7000, 8000, 9000 or 10,000 nucleotides in length. In some embodiments, the transgene is greater than 10 kb in length.

In some embodiments, the coding sequence for each ITAM in the transgene is codon-optimized to not have sequence identity to one another of 12 consecutive nucleotides or longer. In some embodiments, the coding sequence for each ITAM in the transgene is codon-optimized to not have sequence identity to one another of 9 consecutive nucleotides or longer.

In some embodiments, the ITAM is a cytoplasmic signaling sequence of a protein selected from the group consisting of TCRzeta, FcRgamma, FcRbeta, CD3gamma, CD3delta, CD3epsilon, CD5, CD22, CD79a, CD79b and CD66d. In some embodiments, the ITAM is CD3.

Also provided, in another embodiment, is a cell comprising the transposon of the present disclosure. In some embodiments, the cell is a T cell, NK cell, NKT cell, monocyte, macrophage, PBMC, or a precursor cell (e.g., iPSC) thereof.

In some embodiments, the cell further comprises a transposase. In some embodiments, the transposase is selected from the group consisting of a piggyBac® transposase, a piggy-Bac® like transposase, a Super piggyBac® (SPB) transposase, a piggyBat transposase, a Sleeping Beauty transposase, a hyperactive Sleeping Beauty (SB100X) transposase, Helitron transposase, a Tol2 transposase, a TcBuster transposase or a hyperactive TcBuster transposase. In some embodiments, the transposase is Sleeping Beauty transposase SB100X.

Another embodiment provides a method for preparing transfected lymphocytes, comprising obtaining a sample comprising T cells from a donor subject; incubating the sample with a transposon to transfect the T cells to produce transfected T cells; and culturing the sample comprising the transfected T cells for less than 96 hours before the T cells are harvested to produce a harvested sample, wherein at least 40% of T cells in the harvest sample are naïve T cells. In some embodiments, at least 50% of T cells in the harvest sample are naïve T cells.

In some embodiments, the naïve T cells are characterized as CD45RA+ and CCR7+. In some embodiments, the naïve T cells are further characterized as CD62L⁺, CD27⁺ and CD28⁺.

In some embodiments, the T cells are not activated prior to the transfection. In some embodiments, the T cells are not enriched prior to the transfection.

In some embodiments, the transgene is at least 5000 nucleotides in length. In some embodiments, the transgene is at least 6000 nucleotides in length. In some embodiments, the transgene is greater than 10 kb in length. In some embodiments, the transposon comprises a transgene encoding a chimeric antigen receptor (CAR) or T cell receptor (TCR).

In another embodiment, provided is a method for preparing lymphocytes that express a chimeric antigen receptor (CAR) or T cell receptor (TCR), comprising: acquiring a biological sample comprising lymphocytes from a human subject; introducing to the lymphocytes a transposase and a transposon comprising a transgene encoding a polypeptide that comprises the CAR or the TCR; and harvesting lymphocytes comprising the transposase and transposon, wherein the lymphocyte harvesting occurs within 96 hours following the biological sample acquisition.

In some embodiments, the transposase and transposon are introduced to the lymphocytes through physical delivery methods, such as electroporation, nucleofection, lipofection, ultrasound, or magnetofection. In some embodiments, the transposase and transposon are introduced to the lymphocytes through electroporation. Transposase can be delivered into a cell as DNA, mRNA, or protein.

In some embodiments, the lymphocyte harvesting occurs within 48 hours following the biological sample acquisition. In some embodiments, the method further comprises cryopreserving the harvested lymphocytes, or injecting the harvested lymphocytes to a patient, wherein the cryopreservation or injection occurs within 48 hours following the biological sample acquisition.

In some embodiments, the method does not include lymphocyte activation. In some embodiments, the method does not include lymphocyte enrichment. In some embodiments, the method does not include lymphocyte expansion. In some embodiments, the lymphocyte harvesting occurs within 36 hours following the biological sample acquisition.

In some embodiments, the lymphocytes are NK cells. In some embodiments, the lymphocytes are NKT cells. In some embodiments, the lymphocytes are T cells. In some embodiments, at least 40% of the T cells that have the transgene integrated into the genome are naïve T cells when harvested. In some embodiments, the naïve T cells are CD45RA⁺ and CCR7⁺.

In some embodiments, the polypeptide further comprises a second CAR, and the first CAR and the second CAR each comprises a single chain fragment (scFv), a transmembrane domain, and an immunoreceptor tyrosine-based activation motif (ITAM). In some embodiments, the polypeptide comprises a total of three or four CAR each comprising a scFv, a transmembrane domain, and an ITAM. In some embodiments, the transposon comprises two or more transgenes which collectively encode two, three, four or more CAR.

In some embodiments, the transposon is a DNA transposon selected from the group consisting of a Sleeping Beauty transposon, a piggyBac transposon, and a TcBuster transposon, or a retro-transposon. In some embodiments, the transposon is a Sleeping Beauty transposon and the transposase is a Sleeping Beauty transposase. In some embodiments, the transgene is at least 5000 nucleotides in length. In some embodiments, the transgene is greater than 10 kb in length.

In some embodiments, the sample subjected to transfection comprise at least 25×10⁶ cells. In some embodiments, the sample subjected to transfection comprise at least 50×10⁶ cells. In some embodiments, the transfection is conducted in a solution having a volume of 0.5 mL to 2 mL. In some embodiments, the transfection results in at least 40% of T cells or lymphocytes in the sample being transfected.

### BRIEF DESCRIPTION OF THE DRAWINGS

FIG. 1 is a schematic depicting three different plasmid constructs: Plasmid 1 (7725 bp including a 4835 bp insert), Plasmid 2 (8203 bp including a 5313 bp insert), and Plasmid 3 (8132 bp including a 5242 bp insert).

FIG. 2 is a schematic depicting two different large payload plasmid constructs: Plasmid 4 (10929 bp including a 8038 bp insert), and Plasmid 5 (12754 bp including a 9863 bp insert).

FIGS. 3A-3C show plots of CAR expression, at Day 8 post-electroporation with Plasmid 4.

FIGS. 4A-4C show plots of CAR expression, at Day 8 post-electroporation with Plasmid 5.

### DETAILED DESCRIPTION OF THE DISCLOSURE

### Definitions

In order for the present disclosure to be more readily understood, certain terms are first defined below. Additional definitions for the following terms and other terms are set forth throughout the Specification.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specifically stated or evident from context the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

“Administering” refers to the physical introduction of an agent to a subject, such as a modified T cell disclosed herein, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “allogeneic” refers to any material derived from one individual which is then introduced to another individual of the same species, e.g., allogeneic T cell transplantation.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

An “antigen binding molecule,” “antigen binding portion,” “antigen binding fragment,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′) 2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecule. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments an antigen binding molecule is a chimeric antigen receptor (CAR) or an engineered T cell receptor (TCR).

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Chimeric antigen receptor” or “CAR” refers to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR T cell. “Extracellular domain” (or “ECD”) refers to a portion of a polypeptide that, when the polypeptide is present in a cell membrane, is understood to reside outside of the cell membrane, in the extracellular space.

A “T cell receptor” or “TCR” refers to antigen-recognition molecules present on the surface of T cells. During normal T cell development, each of the four TCR genes, α, β, γ, and δ, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

Term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided polypeptide sequences are known. Calculation of the percent identity of two nucleic acid or polypeptide sequences, for example, may be performed by aligning the two sequences for optimal comparison purposes (e.g., gaps may be introduced in one or both of a first and a second sequences for optimal alignment and non-identical sequences may be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally taking into account the number of gaps, and the length of each gap, which may need to be introduced for optimal alignment of the two sequences. Comparison or alignment of sequences and determination of percent identity between two sequences may be accomplished using a mathematical algorithm, such as BLAST (basic local alignment search tool). In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical (e.g., 85-90%, 85-95%, 85-100%, 90-95%, 90-100%, or

The immune cells of the immunotherapy can come from any source known in the art. For example, immune cells can be differentiated in vitro from a hematopoietic stem cell population, or immune cells can be obtained from a subject. Immune cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the immune cells can be derived from one or more immune cell lines available in the art. Immune cells can also be obtained from a unit of blood collected from a subject using any number of techniques known to the skilled artisan, such as FICOLL™ separation and/or apheresis. Additional methods of isolating immune cells for an immune cell therapy are disclosed in U.S. Patent Publication No. 2013/0287748, which is herein incorporated by references in its entirety.

A “patient” includes any human who is afflicted with a cancer (e.g., a lymphoma or a leukemia). The terms “subject” and “patient” are used interchangeably herein.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

The term “shortened autologous process” refers to a transposon-based CAR T-cell manufacturing process which is completed in less than 7 days, less than 5 days, less than 3 days, less than 1 day, less than 18 hours, less than 12 hours, less than 6 hours, less than 4 hours, or less than two hours.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

The terms “transfection” and transfected” refer to the process whereby foreign DNA is introduced into a cell via a non-viral vector. In some embodiments, the non-viral vector is a transposon. In certain embodiments, the transposon is selected from a piggyBac® (PB) transposon, a piggy-Bac® like transposon, a piggyBac transposon, a Sleeping Beauty transposon, a Helraiser transposon, a Tol2 transposon or a TcBuster transposon. In certain further aspects, the transposon can be integrated into the genome of the cell by a corresponding transposase. In certain aspects, the transposase can be a piggyBac® transposase, a piggy-Bac® like transposase, a Super piggyBac® (SPB) transposase, a piggyBat transposase, a Sleeping Beauty transposase, a hyperactive Sleeping Beauty (SB 100X) transposase, Helitron transposase, a Tol2 transposase, a TcBuster transposase or a hyperactive TcBuster transposase, without limitation. The Helitron transposase can be a Helibatl transposase.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

### Non-Viral Vehicles

As provided, production of CAR and TCR cells with viral vectors are associated with high processing costs, inherent safety risks and limitation on the sizes of the coding sequences. It is commonly recognized that retroviral vectors can only carry a payload less than 6 kilobases (KB) in length, and a lentiviral vector may carry a payload slightly longer than 7.5 KB. Such may be sufficient for one regular CAR or TCR coding sequences, but are barely enough for bi-cistronic ones, let alone much needed enhancement elements to further improve the efficacy and safety of the regular CAR or TCR.

Many non-viral approaches are being investigated. The instant inventors, however, demonstrated herein that the transposon systems can overcome various challenges faced by the viral vector-based system. First, the transposon systems can overcome the physical restriction of viral vectors on payload size. Several tested DNA transposons can support payloads larger than 10 kb, allowing incorporation of multiple CARs and/or enhancement elements that may be helpful to boost the therapeutic cell products. In certain aspects, transposon-based systems simplify multi-genetic editing to one step transfection.

Second, when two or more CARs are used together, it is likely that they share long strings of similar sequences. For instance, two CARs in a bi-cistronic CAR may include the same immunoreceptor tyrosine-based activation motif (ITAM) such as CD3 zeta. Such homology can cause recombination, leading to deletion or truncation of either or both the CARs. Such recombination has indeed been observed in lentiviral systems. As the complexity of gene engineering increases, the risk of having recombination issues will also increase.

As Example 1 demonstrates, even when the homology between two copies of CD3 zeta was minimized by codon optimization and sequence wobbling, there was still significant amounts of recombination following lentiviral transduction, as represented by multiple variants of expressed proteins. Use of the transposon system in Example 1, however, led to reduction of such recombination, and total elimination of the most damaging recombination (e.g., variants V1 and V15, with large truncations/deletions; see, e.g., Table 1).

Meanwhile, the transposon systems can serve as an alternative to viral vectors by offering similar transfection efficiency, cell viability and integration stability (see, e.g., Tables 2-8).

Yet another challenge for the conventional viral vector-based autologous cellular therapy is its associated long processing time and high costs. A typical lentiviral vector-based autologous process takes about 7-10 days. Critical steps in such a conventional process include T cell activation, T-cell enrichment and expansion. The instant inventors, however, were able to develop a transposon-based process that can be completed within 24 hours, without the need of T cell activation, T-cell enrichment or expansion.

Surprisingly, such a greatly quicker process, even at lower doses, achieved greater in vivo efficacy than the conventional 7-day lentiviral vector-based process at higher doses (Examples 4 and 5). The data presented in the examples (e.g., Tables 9 and 14) suggest that the shortened process led to harvesting of higher concentrations of naïve T cells which contributed to the improved therapeutic effects.

Also importantly, as shown in Tables 12 and 13, the transfection of these transposons caused relatively modest cell death, and the transfected T cells were able to expand rapidly.

In Example 8, comparison was made between Sleeping Beauty and TcBuster, another transposon system. The results show that both transposon systems were able to generate highly efficacious CAR-T cells. The CAR-T cells prepared with the Sleeping Beauty and TcBuster transposon systems, or with lentiviral vectors, all were able to effectively inhibit tumor growth for at least 20 days in the Nalm6 animal model.

Based on these successful process developments, the instant inventors were also able to develop a large-scale process that utilized 50×10⁶ to 100×10⁶ cells (applicable to billions of cells) in the starting materials (compared to 5×10⁶ in the pilot small scale study). Interestingly, not only did the larger scale not reduce the efficacy or quality of the process, it also actually resulted in significantly improved cell viability and transfection efficacy.

In accordance with one embodiment of the present disclosure, therefore, provided is a transposon that includes a transgene encoding one or more than one polypeptide that includes a chimeric antigen receptor (CAR) or T-cell receptor (TCR). In certain aspects, the transgene encodes two, three, four, five, six, seven, eight, nine or ten different polypeptides, wherein one or more polypeptides is a chimeric antigen receptor (CAR) or T-cell receptor (TCR).

In some embodiments, the transgene is at least 500 nucleotides in length. In some embodiments, the transgene is at least 600, 700, 800, 900, 1,000, 1,500, 2,000, 3,000, 4,000, 5,000 6,000, 7,000, 8,000, 9,000, 10,000, 11,000, 12,000, 13,000, 14,000, 15,000 or 20,000 nucleotides in length. In certain further embodiments, the transgene can be greater than 20,000 nucleotides in length. In some embodiments, the transgene is not longer than 10,000, 11,000, 12,000, 13,000, 14,000, 15,000, 20,000, 25,000 or 30,000 nucleotides in length.

In some embodiments, the transgene includes two fragments sharing homology. In some embodiments, the homology is at least 70% sequence identity. In some embodiments, the homology is at least 75%, 80%, 85%, 90%, 95%, 98% or 99% sequence identity. In some embodiments, the two fragments are each in a different CAR or TCR sequence.

An example of such a fragment is an immunoreceptor tyrosine-based activation motif (ITAM). An ITAM is typically found in cytoplasmic tails of non-catalytic tyrosine-phosphorylated receptors, cell-surface proteins found mainly on immune cells. It is an integral component for the initiation of a variety of signaling pathway and subsequently the activation of immune cells. Non-limiting examples of ITAM include cytoplasmic signaling sequences of proteins such as TCRzeta, FcRgamma, FcRbeta, CD3gamma, CD3delta, CD3epsilon, CD5, CD22, CD79a, CD79b and CD66d. A particular example is CD3.

Such homology can also be found in other portions of the CAR or TCR sequences, such as linkers, extracellular hinges, transmembrane domains, intracellular signaling domains, or even antigen-binding sequences, without limitation.

In some embodiments, the homologous fragments are codon-optimized to reduce sequence identity without altering the encoded proteins. In some embodiments, the homologous fragments are codon-optimized so that they don't share sequence identity of 30, 25, 20, 19, 18, 17, 16, 15, 14, 13, 12, 11, 10, 9, or 8 consecutive nucleotides or longer. In some embodiments, the homologous fragments are codon-optimized so that they don't share sequence identity of 15 consecutive nucleotides or longer. In some embodiments, the homologous fragments are codon-optimized so that they don't share sequence identity of 12 consecutive nucleotides or longer. In some embodiments, the homologous fragments are codon-optimized so that they don't share sequence identity of 10 consecutive nucleotides or longer. In some embodiments, the homologous fragments are codon-optimized so that they don't share sequence identity of 9 consecutive nucleotides or longer. In some embodiments, the homologous fragments are codon-optimized so that they don't share sequence identity of 8 consecutive nucleotides or longer.

In some embodiments, the transgene encodes a first CAR (or TCR) and a second CAR (or TCR), which can optionally be concatenated via a protease cleavage site (e.g., a self-cleavage site) or a ribosomal skip sequence. In some embodiments, the first CAR and the second CAR each includes a single chain fragment (scFv), a transmembrane domain, and an immunoreceptor tyrosine-based activation motif (ITAM).

In some embodiments, the transgene encodes a third CAR (or TCR). In some embodiments, the transgene further encodes a fourth CAR (or TCR). Each of the second, third or third CAR (or TCR) can be cleaved at a protease cleavage site (e.g., a self-cleavage site) or a ribosomal skip sequence to form individual CAR (TCR).

In some embodiments, the transposon includes coding sequences for two, three or four CAR (or TCR) but they don't have to be concatenated. Instead, each coding sequence can have its own promoter or other needed regulatory sequences.

### Transposons

The transposon used in the present technology can be any transposon known in the art, such as a piggyBac® (PB) transposon, a piggy-Bac® like transposon, a piggyBat transposon, a Sleeping Beauty transposon, a Helraiser transposon, a Tol2 transposon or a TcBuster transposon.

A transposon can be integrated into the genome of the cell by a corresponding transposase. The integration can be transient or stable. The transgene expression can be transient or stable dependent on the measuring time. The transposase can be a piggyBac® transposase, a piggy-Bac® like transposase, a Super piggyBac® (SPB) transposase, a piggyBat transposase, a Sleeping Beauty transposase, a hyperactive Sleeping Beauty (SB100X) transposase, Helitron transposase, a Tol2 transposase, a TcBuster transposase or a hyperactive TcBuster transposase, without limitation. The Helitron transposase can be a Helibatl transposase.

When the transposon is a piggyBac® transposon, the transposase can be a piggyBac® transposase or a Super piggyBac® transposase. When the transposon is a piggy-Bac® like transposon, the transposase can be a piggy-Bac® like transposase. When the transposon is a Sleeping Beauty transposon, the transposase can be a Sleeping Beauty transposase. When the transposon is a Helraiser transposon, the transposase can be a Helitron transposase. When the transposon is a Tol2 transposon, the transposase can be a Tol2 transposase. When the transposon is a TcBuster transposon, the transposase can be a TcBuster transposase or a hyperactive TcBuster transposase.

In one embodiment, the transposon is a Sleeping Beauty transposon. The Sleeping Beauty transposon can include a nucleic acid that is flanked at either end by inverted repeats which are recognized by an enzyme having Sleeping Beauty transposase activity. By ‘recognized’ is meant that a Sleeping Beauty transposase is capable of binding to the inverted repeat and then integrating the transposon flanked by the inverted repeat into the genome of a target cell. Representative inverted repeats that may be found in the Sleeping Beauty transposons of the subject methods include those disclosed in WO 98/40510 and WO 99/25817. Of particular interest are inverted repeats that are recognized by a transposase that shares at least about 80% amino acid identity to SEQ ID NO:1 of WO 99/25817.

In some embodiments, each inverted repeat of the transposon includes at least one direct repeat. The transposon element is a linear nucleic acid fragment that can be used as a linear fragment or circularized, for example in a plasmid. Alternatively, vectors such as circular plasmids, minicircle plasmids and nanoplasmids can be used. In certain aspects, the backbone encoding the transposon can be anywhere from 100 bp to 4 kb in length. A transposon carrying a gene of interest can be delivered in various DNA formats (plasmid, plasmid alternative, linear DNA, or ds/ss DNA.) In certain embodiments, there are two direct repeats in each inverted repeat sequence. Examples of direct repeat sequences can be found in WO 98/40510, for instance. A miniplasmid is a supercoiled DNA molecule that is about 4,000 bp in length (e.g., between 3000 bp and 5000 bp). In one embodiment, the plasmid is a linear plasmid. In one embodiment, the plasmid is a circular plasmid. In one embodiment, the plasmid is a nanoplasmid. In one embodiment, the plasmid is a minicircle plasmid. As demonstrated in the examples, minicircle plasmids are helpful in achieving high transfection efficiency.

The Sleeping Beauty transposon can be integrated to a target genome by a corresponding transposase, such as SB 10 and SB100X, which are described in, e.g., WO 2017/158029.

### CAR and TCR Sequences

Examples of CAR and TCR sequences are also provided. A CAR includes an antigen-binding portion which typically is a single chain fragment (scFv) derived from an antibody. A CAR may be mono-specific, bi-specific, or multi-specific. In certain aspects, transposon-based systems simplify multi-genetic editing to one step transfection. Also, a construct (or payload) carried by a transposon of the present disclosure may include two or more CAR molecules. A particular example is a bi-cistronic CAR, where two CARs are connected through a digestible linker or a ribosomal skip sequence. Likewise, a bi-cistronic TCR can be included in the payload as well. In certain aspects, a construct (or payload) carried by a transposon of the present disclosure may encode three CARs. In certain aspects, a construct (or payload) carried by a transposon of the present disclosure may encode four or more CARs. In certain further aspects, a construct (or payload) carried by a transposon of the present disclosure may further encode polypeptides which enhance the cytotoxic potential of T-cells expressing one or more CAR.

In certain embodiments, a bi-cistronic, tri-cistronic, or quad-cistronic CAR encoding construct includes a first CAR targeting a first antigen and a second CAR targeting a second antigen. The first and second antigen may be selected from 5T4, alphafetoprotein, B cell maturation antigen (BCMA), CA-125, carcinoembryonic antigen, CD19, CD20, CD22, CD23, CD30, CD33, CD56, CD123, CD138, c-Met, CSPG4, C-type lectin-like molecule 1 (CLL-1), EGFRvIII, epithelial tumor antigen, ERBB2, FLT3, folate binding protein, GD2, GD3, HER1-HER2 in combination, HER2-HER3 in combination, HER2/Neu, HERV-K, HIV-1 envelope glycoprotein gp41, HIV-1 envelope glycoprotein gp120, IL-11Ralpha, kappa chain, lambda chain, melanoma-associated antigen, mesothelin, MUC-1, mutated p53, mutated ras, prostate-specific antigen, ROR1, VEGFR2, or a combination thereof.

In some embodiments, a first, second, third, or fourth CAR targeted antigen is one of 2B4 (CD244), 4-IBB, 5T4, A33 antigen, adenocarcinoma antigen, adrenoceptor beta 3 (ADRB3), A kinase anchor protein 4 (AKAP-4), alpha-fetoprotein (AFP), anaplastic lymphoma kinase (ALK), Androgen receptor, B7H3 (CD276), 02-integrins, BAFF, B-lymphoma cell, B cell maturation antigen (BCMA), bcr-abl (oncogene fusion protein consisting of breakpoint cluster region (BCR) and Abelson murine leukemia viral oncogene homolog 1 (Abl), BhCG, bone marrow stromal cell antigen 2 (BST2), CCCTC-Binding Factor (Zinc Finger Protein)-Like (BORIS or Brother of the Regulator of Imprinted Sites), BST2, C242 antigen, 9-0-acetyl-CA19-9 marker, CA-125, CAEX, calreticulin, carbonic anhydrase 9 (CAIX), C-MET, CCR4, CCR5, CCR8, CD2, CD3, CD4, CD5, CD8, CD7, CD10, CD16, CD19, CD20, CD22, CD23 (IgE receptor), CD24, CD25, CD27, CD28, CD30 (TNFRSF8), CD33, CD34, CD38, CD40, CD40L, CD41, CD44, CD44V6, CD49f, CD51, CD52, CD56, CD63, CD70, CD72, CD74, CD79a, CD79b, CD80, CD84, CD96, CD97, CD100, CD123, CD125, CD133, CD137, CD138, CD150, CD152 (CTLA-4), CD160, CD171, CD179a, CD200, CD221, CD229, CD244, CD272 (BTLA), CD274 (PDL-1, B7H1), CD279 (PD-1), CD352, CD358, CD300 molecule-like family member f (CD300LF), Carcinoembryonic antigen (CEA), claudin 6 (CLDN6), C-type lectin-like molecule-1 (CLL-1 or CLECL1), C-type lectin domain family 12 member A (CLEC12A), a cytomegalovirus (CMV) infected cell antigen, CNT0888, CRTAM (CD355), CS-1 (also referred to as CD2 subset 1, CRACC, CD319, and 19A24), CTLA-4, Cyclin B 1, chromosome X open reading frame 61 (CXORF61), Cytochrome P450 1B 1 (CYP1B1), DNAM-1 (CD226), desmoglein 4, DR3, DRS, E-cadherin neoepitope, epidermal growth factor receptor (EGFR), EGF1R, epidermal growth factor receptor variant III (EGFRvIII), epithelial glycoprotein-2 (EGP-2), epithelial glycoprotein-40 (EGP-40), EGF-like module-containing mucin-like hormone receptor-like 2 (EMR2), elongation factor 2 mutated (ELF2M), endosialin, Epithelial cell adhesion molecule (EPCAM), ephrin type-A receptor 2 (EphA2), Ephrin B2, receptor tyrosine-protein kinases erb-B2,3,4 (erb-B2,3,4), ERBB, ERBB2 (Her2/neu), ERG (transmembrane protease, serine 2 (TMPRSS2) ETS fusion gene), ETA, ETS translocation-variant gene 6, located on chromosome 12p (ETV6-AML), Fc fragment of IgA receptor (FCAR or CD89), fibroblast activation protein alpha (FAP), FBP, Fc receptor-like 5 (FCRL5), fetal acetylcholine receptor (AChR), fibronectin extra domain-B, Fms-Like Tyrosine Kinase 3 (FLT3), folate-binding protein (FBP), folate receptor 1, folate receptor a, Folate receptor (3, Fos-related antigen 1, Fucosyl, Fucosyl GM1; GM2, ganglioside G2 (GD2), ganglioside GD3 (aNeu5Ac(2-8)aNeu5Ac(2-3)bDGalp(1-4)bDG1cp(1-1)Cer),o-acetyl-GD2 ganglioside (OAcGD2), GITR (TNFRSF 18), GM1, ganglioside GM3 (aNeu5Ac(2-3)bDGalp(1-4)bDG1cp(1-1)Cer), GP 100, hexasaccharide portion of globoH glycoceramide (GloboH), glycoprotein 75, Glypican-3 (GPC3), glycoprotein 100 (gp100), GPNMB, G protein-coupled receptor 20 (GPR20), G protein-coupled receptor class C group 5, member D (GPRCSD), Hepatitis A virus cellular receptor 1 (HAVCR1), human Epidermal Growth Factor Receptor 2 (HER-2), HER2/neu, HER3, HER4, HGF, high molecular weight-melanoma-associated antigen (HMWMAA), human papilloma virus E6 (HPV E6), human papilloma virus E7 (HPV E7), heat shock protein 70-2 mutated (mut hsp70-2), human scatter factor receptor kinase, human Telomerase reverse transcriptase (hTERT), HVEM, ICOS, insulin-like growth factor receptor 1 (IGF-1 receptor), IGF-I, IgGl, immunoglobulin lambda-like polypeptide 1 (IGLL1), IL-6, Interleukin 11 receptor alpha (IL-11Ra), IL-13, Interleukin-13 receptor subunit alpha-2 (IL-13Ra2 or CD213A2), insulin-like growth factor I receptor (IGF1-R), integrin α5β1, integrin ανβ3, intestinal carboxyl esterase, κ-light chain, KCS1, kinase insert domain receptor (KDR), KIR, KIR2DL1, KIR2DL2, KIR2DL3, KIR3DL2, KIR-L, KG2D ligands, KIT (CD117), KLRGI, LAGE-1a, LAG3, lymphocyte-specific protein tyrosine kinase (LCK), Leukocyte immunoglobulin-like receptor subfamily A member 2 (LILRA2), legumain, Leukocyte-associated immunoglobulin-like receptor 1 (LAIR1), Lewis(Y) antigen, LeY, LG, LI cell adhesion molecule (LI-CAM), LIGHT, LMP2, lymphocyte antigen 6 complex, LTBR, locus K 9 (LY6K), Ly-6, lymphocyte antigen 75 (LY75), melanoma cancer testis antigen-1 (MAD-CT-1); melanoma cancer testis antigen-2 (MAD-CT-2), MAGE, Melanoma-associated antigen 1 (MAGE-A1), MAGE-A3 melanoma antigen recognized by T cells 1 (MelanA or MARTI), MelanA/MARTI, Mesothelin, MAGE A3, melanoma inhibitor of apoptosis (ML-IAP), melanoma-specific chondroitin-sulfate proteoglycan (MCSCP), MORAb-009, MS4A1, Mucin 1 (MUC1), MUC2, MUC3, MUC4, MUCSAC, MUC5b, MUC7, MUC16, mucin CanAg, Mullerian inhibitory substance (MIS) receptor type II, v-myc avian myelocytomatosis viral oncogene neuroblastoma derived homolog (MYCN), N-glycolylneuraminic acid, N-Acetyl glucosaminyl-transferase V (NA17), neural cell adhesion molecule (NCAM), NKG2A, NKG2C, NKG2D, NKG2E ligands, NKR-P IA, NPC-1C, NTB-A, mammary gland differentiation antigen (NY-BR-1), NY-ESO-1, oncofetal antigen (h5T4), Olfactory receptor 51E2 (OR51E2), OX40, plasma cell antigen, poly SA, proacrosin binding protein sp32 (OY-TES 1), p53, p53 mutant, pannexin 3 (PANX3), prostatic acid phosphatase (PAP), paired box protein Pax-3 (PAX3), Paired box protein Pax-5 (PAX5), prostate carcinoma tumor antigen-1 (PCTA-1 or Galectin 8), PD-1H, Platelet-derived growth factor receptor alpha (PDGFR-alpha), PDGFR-beta, PDL192, PEN-5, phosphatidylserine, placenta-specific 1 (PLAC1), Polysialic acid, Prostase, prostatic carcinoma cells, prostein, Protease Serine 21 (Testisin or PRSS21), Proteinase3 (PR1), prostate stem cell antigen (PSCA), prostate-specific membrane antigen (PSMA), Proteasome (Prosome, Macropain) Subunit, Beta Type, Receptor for Advanced Glycation Endproducts (RAGE-1), RANKL, Ras mutant, Ras Homolog Family Member C (RhoC), RON, Receptor tyrosine kinase-like orphan receptor 1 (ROR1), renal ubiquitous 1 (RU1), renal ubiquitous 2 (RU2), sarcoma translocation breakpoints, Squamous Cell Carcinoma Antigen Recognized By T Cells 3 (SART3), SAS, SDC1, SLAMF7, sialyl Lewis adhesion molecule (sLe), Siglec-3, Siglec-7, Siglec-9, sonic hedgehog (SHH), sperm protein 17 (SPA17), Stage-specific embryonic antigen-4 (SSEA-4), STEAP, sTn antigen, synovial sarcoma, X breakpoint 2 (SSX2), Survivin, Tumor-associated glycoprotein 72 (TAG72), TCR5y, TCRa, TCRB, TCR Gamma Alternate Reading Frame Protein (TARP), telomerase, TIGIT TNF-α precursor, tumor endothelial marker 1 (TEM1/CD248), tumor endothelial marker 7-related (TEM7R), tenascin C, TGF beta 2, TGF-β, transglutaminase 5 (TGSS), angiopoietin-binding cell surface receptor 2 (Tie 2), TIM1, TIM2, TIM3, Tn Ag, TRAIL-R1, TRAIL-R2, Tyrosinase-related protein 2 (TRP-2), thyroid stimulating hormone receptor (TSHR), tumor antigen CTAA16.88, Tyrosinase, ROR1, TAG-72, uroplakin 2 (UPK2), VEGF-A, VEGFR-1, vascular endothelial growth factor receptor 2 (VEGFR2), and vimentin, Wilms tumor protein (WT1), or X Antigen Family, Member 1A (XAGE1).

In one embodiment, the first antigen and the second antigen are CD19 and CD20, respectively. Example sequences of anti-CD19 and anti-CD20 antibodies and fragments, as well as their bi-cistronic versions are provided in Table A.

TABLE A
Example Antigen-Binding Sequences

| Name | Sequence |
|---|---|
| Anti-CD20 v01 | SEQ ID NO: 1 |
| VH/VL | QVQLQQWGAGLLKPSETLSLTCAVYGGSFSGYYWSWIRQPPGKGLEWIGEIDH |
| | SGSTNYNPSLKSRVTISVDTSKNQFSLKLSSVTAADTAVYYCARGGGSWYSNW |
| | FDPWGQGTMVTVSS |
| | SEQ ID NO: 2 |
| | DIQMTQSPSTLSASVGDRVTITCRASQSISSWLAWYQQKPGKAPKLLIYDASS |
| | LESGVPSRFSGSGSGTEFTLTISSLQPDDFATYYCQQDRSLPPTFGGGTKVEI |
| | K |
| Anti-CD20 v02 | SEQ ID NO: 3 |
| VH/VL | QVQLQQWGAGLLKPSETLSLTCAVYGGSFSGIHWNWIRQPPGKGLEWIGDIDT |
| | SGSTNYNPSLKSRVTISVDTSKNQFSLKLSSVTAADTAVYYCARLGQESATYL |
| | GMDVWGQGTTVTVSS |
| | SEQ ID NO: 4 |
| | DIVMTQSPDSLAVSLGERATINCKSSQSVLYSSNNKNYLAWYQQKPGQPPKLL |
| | IYWASTRESGVPDRFSGSGSGTDFTLTISSLQAEDVAVYYCQQLYTYPFTFGG |
| | GTKVEIK |
| Anti-CD20 v03 | SEQ ID NO: 5 |
| VH/VL | QLQLQESGPGLVKPSETLSLTCTVSGGSISSSSYYWGWIRQPPGKGLEWIGSI |
| | YYSGSTYYNPSLKSRVTISVDTSKNQFSLKLSSVTAADTAVYYCARETDYSSG |
| | MGYGMDVWGQGTTVTVSS |
| | SEQ ID NO: 6 |
| | DIQMTQSPSSLSASVGDRVTITCRASQSINSYLNWYQQKPGKAPKLLIYAASS |
| | LQSGVPSRFSGSGSGTDFTLTISSLQPEDFATYYCQQSLADPFTFGGGTKVEI |
| | K |
| Anti-CD20 v04 | SEQ ID NO: 7 |
| VH/VL | QVQLVQSGAEVKKPGASVKVSCKASGYTFKEYGISWVRQAPGQGLEWMGWISA |
| | YSGHTYYAQKLQGRVTMTTDTSTSTAYMELRSLRSDDTAVYYCARGPHYDDWS |
| | GFIIWFDPWGQGTLVTVSS |
| | SEQ ID NO: 8 |
| | DIQMTQSPSSLSASVGDRVTITCRASQSISSYLNWYQQKPGKAPKLLIYAASS |
| | LQSGVPSRFSGSGSGTDFTLTISSLQPEDFATYYCQQSYRFPPTFGQGTKVEI |
| | K |
| Anti-CD20 v05 | SEQ ID NO: 9 |
| VH/VL | QVQLQESGPGLVKPSETLSLTCTVSGGSISSPDHYWGWIRQPPGKGLEWIGSI |
| | YASGSTFYNPSLKSRVTISVDTSKNQFSLKLSSVTAADTAVYYCARETDYSSG |
| | MGYGMDVWGQGTTVTVSS |
| | SEQ ID NO: 10 |
| | DIQMTQSPSSLSASVGDRVTITCRASQSINSYLNWYQQKPGKAPKLLIYAASS |
| | LQSGVPSRFSGSGSGTDFTLTISSLQPEDFATYYCQQSLADPFTFGGGTKVEI |
| | K |
| Anti-CD20 v06 | SEQ ID NO: 11 |
| VH/VL | QITLKESGPTLVKPTQTLTLTCTFSGFSLDTEGVGVGWIRQPPGKALEWLALI |
| | YFNDQKRYSPSLKSRLTITKDTSKNQVVLTMINMDPVDTAVYYCARDTGYSRW |
| | YYGMDVWGQGTTVTVSS |
| | SEQ ID NO: 12 |
| | DIQMTQSPSSVSASVGDRVTITCRASQGISSWLAWYQQKPGKAPKLLIYAASS |
| | LQSGVPSRFSGSGSGTDFTLTISSLQPEDFATYYCQQAYAYPITFGGGTKVEI |
| | K |
| Anti-CD20 v07 | SEQ ID NO: 13 |
| VH/VL | QVQLQQWGAGLLKPSETLSLTCAVYGGSFEKYYWSWIRQPPGKGLEWIGEIYH |
| | SGLTNYNPSLKSRVTISVDTSKNQFSLKLSSVTAADTAVYYCARVRYDSSDSY |
| | YYSYDYGMDVWGQGTTVTVSS |
| | SEQ ID NO: 14 |
| | DIVLTQSPDSLAVSLGERATINCKSSQSVLYSSNNKNYLAWYQQKPGQPPKLL |
| | IYWASSRESGVPDRFSGSGSGTDFTLTISSLQAEDVAVYYCQQSYSFPWTFGG |
| | GTKVEIK |
| Anti-CD20 v08 | SEQ ID NO: 15 |
| VH/VL | QVQLQQWGAGLLKPSETLSLTCAVYGGSFSRYVWSWIRQPPGKGLEWIGEIDS |
| | SGKTNYNPSLKSRVTISVDTSKNQFSLKLSSVTAADTAVYYCARVRYDSSDSY |
| | YYSYDYGMDVWGQGTTVTVSS |
| | SEQ ID NO: 16 |
| | DIVLTQSPDSLAVSLGERATINCKSSQSVLYSSNNKNYLAWYQQKPGQPPKLL |
| | IYWASSRESGVPDRFSGSGSGTDFTLTISSLQAEDVAVYYCQQSYSFPWTFGG |
| | GTKVEIK |
| Anti-CD20 v09 | SEQ ID NO: 17 |
| VH/VL | QVQLQQWGAGLLKPSETLSLTCAVYGGSFSGYAWSWIRQPPGKGLEWIGEIDH |
| | RGFTNYNPSLKSRVTISVDTSKNQFSLKLSSVTAADTAVYYCARVRYDSSDSY |
| | YYSYDYGMDVWGQGTTVTVSS |
| | SEQ ID NO: 18 |
| | DIVLTQSPDSLAVSLGERATINCKSSQSVLYSSNNKNYLAWYQQKPGQPPKLL |
| | IYWASSRESGVPDRFSGSGSGTDFTLTISSLQAEDVAVYYCQQSYSFPWTFGG |
| | GTKVEIK |
| Anti-CD20 v10 | SEQ ID NO: 19 |
| VH/VL | QVQLQQWGAGLLKPSETLSLTCAVYGGSFQKYYWSWIRQPPGKGLEWIGEIDT |
| | SGFTNYNPSLKSRVTISVDTSKNQFSLKLSSVTAADTAVYYCARVGRYSYGYY |
| | ITAFDIWGQGTTVTVSS |
| | SEQ ID NO: 20 |
| | DIVMTQSPDSLAVSLGERATINCKSSQSVLYSSNNKNYLAWYQQKPGQPPKLL |
| | IYWASTRESGVPDRFSGSGSGTDFTLTISSLQAEDVAVYYCQQHYSFPFTFGG |
| | GTKVEIK |
| Anti-CD19 | SEQ ID NO: 21 |
| VH/VL v01 | EVKLQESGPGLVAPSQSLSVTCTVSGVSLPDYGVSWIRQPPRKGLEWLGVIWG |
| | SETTYYNSALKSRLTIIKDNSKSQVFLKMNSLQTDDTAIYYCAKHYYYGGSYA |
| | MDYWGQGTSVTVSS |
| | SEQ ID NO: 22 |
| | DIQMTQTTSSLSASLGDRVTISCRASQDISKYLNWYQQKPDGTVKLLIYHTSR |
| | LHSGVPSRFSGSGSGTDYSLTISNLEQEDIATYFCQQGNTLPYTFGGGTKLEI |
| | T |
| Anti-CD19 | SEQ ID NO: 23 |
| VH/VL v02 | EVQLVESGGGLVQPGRSLRLSCTASGVSLPDYGVSWIRQPPGKGLEWIGVIWG |
| | SETTYYNSALKSRFTISRDNSKNTLYLQMNSLRAEDTAVYYCAKHYYYGGSYA |
| | MDYWGQGTLVTVSS |
| | SEQ ID NO: 24 |
| | DIQMTQSPSSLSASVGDRVTITCRASQDISKYLNWYQQKPDQAPKLLIKHTSR |
| | LHSGVPSRFSGSGSGTDYTLTISSLQPEDFATYYCQQGNTLPYTFGQGTKLEI |
| | K |
| Anti-CD19 scFv | SEQ ID NO: 25 |
| | DIQMTQTTSSLSASLGDRVTISCRASQDISKYLNWYQQKPDGTVKLLIYHTSR |
| | LHSGVPSRFSGSGSGTDYSLTISNLEQEDIATYFCQQGNTLPYTFGGGTKLEI |
| | TGSTSGSGKPGSGEGSTKGEVKLQESGPGLVAPSQSLSVTCTVSGVSLPDYGV |
| | SWIRQPPRKGLEWLGVIWGSETTYYNSALKSRLTIIKDNSKSQVFLKMNSLQT |
| | DDTAIYYCAKHYYYGGSYAMDYWGQGTSVTVSS |
| Anti-CD20/anti- | SEQ ID NO: 26 |
| CD19 bi- | MLLLVTSLLLCELPHPAFLLIPDIQMTQTTSSLSASLGDRVTISCRASQDISK |
| cistronic CAR | YLNWYQQKPDGTVKLLIYHTSRLHSGVPSRFSGSGSGTDYSLTISNLEQEDIA |
| v01 | TYFCQQGNTLPYTFGGGTKLEITGGGGSGGGGSGGGGSEVKLQESGPGLVAPS |
| | QSLSVTCTVSGVSLPDYGVSWIRQPPRKGLEWLGVIWGSETTYYNSALKSRLT |
| | IIKDNSKSQVFLKMNSLQTDDTAIYYCAKHYYYGGSYAMDYWGQGTSVTVSSA |
| | AALDNEKSNGTIIHVKGKHLCPSPLFPGPSKPFWVLVVVGGVLACYSLLVTVA |
| | FIIFWVRSKRSRLLHSDYMNMTPRRPGPTRKHYQPYAPPRDFAAYRSRVKFSR |
| | SADAPAYQQGQNQLYNELNLGRREEYDVLDKRRGRDPEMGGKPRRKNPQEGLY |
| | NELQKDKMAEAYSEIGMKGERRRGKGHDGLYQGLSTATKDTYDALHMQALPPR |
| | RAKRSGSGEGRGSLLTCGDVEENPGPMALPVTALLLPLALLLHAARPQLQLQE |
| | SGPGLVKPSETLSLTCTVSGGSISSSSYYWGWIRQPPGKGLEWIGSIYYSGST |
| | YYNPSLKSRVTISVDTSKNQFSLKLSSVTAADTAVYYCARETDYSSGMGYGMD |
| | VWGQGTTVTVSSGSTSGSGKPGSGEGSTKGDIQMTQSPSSLSASVGDRVTITC |
| | RASQSINSYLNWYQQKPGKAPKLLIYAASSLQSGVPSRFSGSGSGTDFTLTIS |
| | SLQPEDFATYYCQQSLADPFTFGGGTKVEIKAAAFVPVFLPAKPTTTPAPRPP |
| | TPAPTIASQPLSLRPEACRPAAGGAVHIRGLDFACDIYIWAPLAGTCGVLLLS |
| | LVITLYCNHRNRFSVVKRGRKKLLYIFKQPFMRPVQTTQEEDGCSCRFPEEEE |
| | GGCELRVKFSRSADAPAYQQGQNQLYNELNLGRREEYDVLDKRRGRDPEMGGK |
| | PRRKNPQEGLYNELQKDKMAEAYSEIGMKGERRRGKGHDGLYQGLSTATKDTY |
| | DALHMQALPPR |
| Anti-CD20/anti- | SEQ ID NO: 27 |
| CD19 bi- | MLLLVTSLLLCELPHPAFLLIPDIQMTQSPSSLSASVGDRVTITCRASQSINS |
| cistronic CAR | YLNWYQQKPGKAPKLLIYAASSLQSGVPSRFSGSGSGTDFTLTISSLQPEDFA |
| v02 | TYYCQQSLADPFTFGGGTKVEIKGGGGSGKPGSGEGGSQLQLQESGPGLVKPS |
| | ETLSLTCTVSGGSISSSSYYWGWIRQPPGKGLEWIGSIYYSGSTYYNPSLKSR |
| | VTISVDTSKNQFSLKLSSVTAADTAVYYCARETDYSSGMGYGMDVWGQGTTVT |
| | VSSGGGGSGKPGSDIQMTQSPSSLSASVGDRVTITCRASQDISKYLNWYQQKP |
| | DQAPKLLIKHTSRLHSGVPSRFSGSGSGTDYTLTISSLQPEDFATYYCQQGNT |
| | LPYTFGQGTKLEIKGGGGSGGGGSGGGGSEVQLVESGGGLVQPGRSLRLSCTA |
| | SGVSLPDYGVSWIRQPPGKGLEWIGVIWGSETTYYNSALKSRFTISRDNSKNT |
| | LYLQMNSLRAEDTAVYYCAKHYYYGGSYAMDYWGQGTLVTVSSAAALDNEKSN |
| | GTIIHVKGKHLCPSPLFPGPSKPFWVLVVVGGVLACYSLLVTVAFIIFWVRSK |
| | RSRLLHSDYMNMTPRRPGPTRKHYQPYAPPRDFAAYRSRVKFSRSADAPAYQQ |
| | GQNQLYNELNLGRREEYDVLDKRRGRDPEMGGKPRRKNPQEGLYNELQKDKMA |
| | EAYSEIGMKGERRRGKGHDGLYQGLSTATKDTYDALHMQALPPR |

A CAR of the present disclosure can include, in addition to the antigen-binding molecule, a hinge, a transmembrane domain, and/or an intracellular domain. In some embodiments, the intracellular domain can include a costimulatory domain and an activation domain.

A hinge may be an extracellular domain of an antigen binding system positioned between the binding motif and the transmembrane domain. A hinge may also be referred to as an extracellular domain or as a “spacer.” A hinge may contribute to receptor expression, activity, and/or stability. A hinge may also provide flexibility to access the targeted antigen. In some embodiments, a hinge domain is positioned between a binding motif and a transmembrane domain.

In some embodiments, the hinge is, is from, or is derived from (e.g., comprises all or a fragment of) an immunoglobulin-like hinge domain. In some embodiments, a hinge domain is from or derived from an immunoglobulin. In some embodiments, a hinge domain is selected from the hinge of IgG1, IgG2, IgG3, IgG4, IgA, IgD, IgE, or IgM, or a fragment thereof.

In some embodiments, the hinge is, is from, or is derived from (e.g., comprises all or a fragment of) CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, or which is a fragment or combination thereof.

In some embodiments, the hinge is, is from, or is derived from (e.g., comprises all or a fragment of) a hinge of CD8 alpha. In some embodiments, the hinge is, is from, or is derived from a hinge of CD28. In some embodiments, the hinge is, is from, or is derived from a fragment of a hinge of CD8 alpha or a fragment of a hinge of CD28, wherein the fragment is anything less than the whole. In some embodiments, a fragment of a CD8 alpha hinge or a fragment of a CD28 hinge comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of a CD8 alpha hinge, or of a CD28 hinge.

A “transmembrane domain” refers to a domain having an attribute of being present in the membrane when present in a molecule at a cell surface or cell membrane (e.g., spanning a portion or all of a cellular membrane). It is not required that every amino acid in a transmembrane domain be present in the membrane. For example, in some embodiments, a transmembrane domain is characterized in that a designated stretch or portion of a protein is substantially located in the membrane. Amino acid or nucleic acid sequences may be analyzed using a variety of algorithms to predict protein subcellular localization (e.g., transmembrane localization). The programs psort (PSORT.org) and Prosite (prosite.expasy.org) are exemplary of such programs.

A transmembrane domain may be derived either from any membrane-bound or transmembrane protein, such as an alpha, beta or zeta chain of a T-cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CD5, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

The intracellular domain (or cytoplasmic domain) comprises one or more signaling domains that, upon binding of target antigen to the binding motif, cause and/or mediate an intracellular signal, e.g., that activates one or more immune cell effector functions (e.g., native immune cell effector functions). In some embodiments, signaling domains of an intracellular domain mediate activation at least one of the normal effector functions of the immune cell. Effector function of a T cell, for example, may be cytolytic activity or helper activity comprising the secretion of cytokines. In some embodiments, signaling domains of an intracellular domain mediate T cell activation, proliferation, survival, and/or other T cell function. An intracellular domain may comprise a signaling domain that is an activating domain. An intracellular domain may comprise a signaling domain that is a costimulatory signaling domain.

Intracellular signaling domains that may transduce a signal upon binding of an antigen to an immune cell are known. For example, cytoplasmic sequences of a T cell receptor (TCR) are known to initiate signal transduction following TCR binding to an antigen (see, e.g., Brownlie et al., Nature Rev. Immunol. 13:257-269 (2013)).

In certain embodiments, suitable signaling domains include, without limitation, those of 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CD5, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

A CAR can also include a costimulatory signaling domain, e.g., to increase signaling potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). Signals generated through a TCR alone may be insufficient for full activation of a T cell and a secondary or co-stimulatory signal may increase activation. Thus, in some embodiments, a signaling domain further comprises one or more additional signaling domains (e.g., costimulatory signaling domains) that activate one or more immune cell effector functions (e.g., a native immune cell effector function described herein). In some embodiments, a portion of such costimulatory signaling domains may be used, as long as the portion transduces the effector function signal. In some embodiments, a cytoplasmic domain described herein comprises one or more cytoplasmic sequences of a T cell co-receptor (or fragment thereof). Non-limiting examples of such T cell co-receptors comprise CD27, CD28, 4-IBB (CD137), OX40, CD30, CD40, PD-1, ICOS, lymphocyte function-associated antigen-1 (LFA-1), MYD88, CD2, CD7, LIGHT, NKG2C, B7-H3, and a ligand that binds with CD83. An exemplary costimulatory protein has the amino acid sequence of a costimulatory protein found naturally on T cells, the complete native amino acid sequence of which costimulatory protein is described in NCBI Reference Sequence: NP 0.1. In certain instances, a CAR includes a 4-IBB costimulatory domain. In certain instances, a CAR includes a CD28 costimulatory domain. In certain instances, a CAR includes a DAP-10 costimulatory domain.

In some embodiments, the CAR further includes an ITAM. Examples of ITAM containing primary cytoplasmic signaling sequences that are of particular use in the disclosure include those derived from TCRzeta, FcRgamma, FcRbeta, CD3gamma, CD3delta, CD3epsilon, CD5, CD22, CD79a, CD79b and CD66d. In some embodiments, the ITAM includes CD3 zeta.

### Transfection, Integration and Expression of Receptors

The transposons of the present disclosure can be introduced to a target cell, in particular immune cells, to generate engineered cells. A target cell is preferably an immune cell, such as T cell, NK cell, monocyte, macrophage, or a precursor cell thereof.

Integration of a transposon to the genome in the target cell requires the expression of a corresponding transposase. The transposon used in the present technology can be any transposon known in the art, such as a piggyBac® (PB) transposon, a piggy-Bac® like transposon, a piggyBat transposon, a Sleeping Beauty transposon, a Helraiser transposon, a Tol2 transposon or a TcBuster transposon.

When the transposon is a piggyBac® transposon, the transposase can be a piggyBac® transposase or a Super piggyBac® transposase. When the transposon is a piggy-Bac® like transposon, the transposase can be a piggy-Bac® like transposase. When the transposon is a Sleeping Beauty transposon, the transposase can be a Sleeping Beauty transposase, such as SB10 or SB100X. When the transposon is a Helraiser transposon, the transposase can be a Helitron transposase. When the transposon is a Tol2 transposon, the transposase can be a Tol2 transposase. When the transposon is a TcBuster transposon, the transposase can be a TcBuster transposase or a hyperactive TcBuster transposase.

The transposase may be introduced to the target cell as a polynucleotide that encodes the transposase, regulated by a promoter that allows its expression in the target cell.

In some embodiments, the transposon and the transposase are introduced to the target cell by a method known in the art, such as electroporation, nucleofection, lipofection, ultrasound, and magnetofection. In some embodiments, the transposon and the transposase are introduced to the target cell by electroporation. In some embodiments, the molecular copy number ratio of the nucleic acid transposon backbone containing the insert of interest and the nucleic acid encoding the transposase is 1:1. In certain further aspects, the molecular copy number ratio of the nucleic acid transposon backbone containing the insert of interest and the nucleic acid encoding the transposase ranges anywhere between 1:1 and 1:32. In some embodiments, the nucleic acid transposon backbone containing the insert of interest is matched in number of copies to nucleic acid encoding the transposase. In certain aspects, the ratio of the nucleic acid transposon backbone containing the insert of interest and the nucleic acid encoding the transposase is 1:4, 1:8, or 1:16.

Upon integration into the target genome, the transgene from the transposon can be transcribed and translated to produce the receptor proteins. As demonstrated in the accompanying experimental examples, such integrated transgenes are highly stable.

### Cell Preparation Processes

The presently disclosed transposon-based technology enabled the instant inventors to develop a cell therapy process that is greatly shorter than the conventional technology. This process is preferably an autologous cell therapy process, but is applicable to an allogenic one as well.

In an example embodiment, the process entails (1) acquiring and/or enriching a population of lymphocytes obtained from a donor subject; (2) transfecting the population of lymphocytes with a transposon of the present disclosure, along with a corresponding transposase; and (3) harvesting transfected lymphocytes. In some embodiments, the harvested lymphocytes are administered to a patient, such as the donor. In some embodiments, the harvested lymphocytes are cryopreserved.

In another embodiment, the process entails obtaining a sample comprising lymphocytes (e.g., T cells) from a donor subject; incubating the sample with a transposon to transfect the lymphocytes to produce transfected lymphocytes; and culturing the sample comprising the transfected lymphocytes before the lymphocytes are harvested to produce a harvested sample, wherein at least 40% of lymphocytes in the harvest sample are naïve cells.

In some embodiments, at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, or 65% of lymphocytes in the harvested sample are naïve cells. In some embodiments, at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, or 65% of T cells in the harvested sample are naïve T cells.

In some embodiments, a naïve T cell is characterized with one or more markers such as CD45RA⁺, CCR7⁺, CD62L⁺, CD27⁺, CD28⁺, CD127⁺, CD132⁺, CD25⁻, CD44⁻, CD45RO⁻, and HLA-DR⁻. In one embodiment, a naïve T cell is characterized with CD45RA⁺ and CCR7⁺. In one embodiment, a naïve T cell is characterized with CD45RA⁺, CCR7⁺, CD62L⁺, CD27⁺, and CD28⁺. In one embodiment, a naïve T cell is characterized additionally with CD127⁺, CD132⁺, CD25⁻, CD44⁻, CD45RO⁻, and/or HLA-DR⁻.

In some embodiments, the entire process, from acquisition of the lymphocytes from the donor to harvesting of the transfected lymphocytes, is completed within 4 days. In some embodiments, the entire process, from acquisition of the lymphocytes from the donor to harvesting of the transfected lymphocytes, is completed within 3 days. In some embodiments, the entire process, from acquisition of the lymphocytes from the donor to harvesting of the transfected lymphocytes, is completed within 2 days. In some embodiments, the entire process, from acquisition of the lymphocytes from the donor to harvesting of the transfected lymphocytes, is completed within 36 hours. In some embodiments, the entire process, from acquisition of the lymphocytes from the donor to harvesting of the transfected lymphocytes, is completed within 24 hours. In some embodiments, the entire process, from acquisition of the lymphocytes from the donor to harvesting of the transfected lymphocytes, is completed within 18 hours. In some embodiments, the entire process, from acquisition of the lymphocytes from the donor to harvesting of the transfected lymphocytes, is completed within 12 hours. In some embodiments, the entire process, from acquisition of the lymphocytes from the donor to harvesting of the transfected lymphocytes, is completed within 8 hours. In some embodiments, the entire process, from acquisition of the lymphocytes from the donor to harvesting of the transfected lymphocytes, is completed within 6 hours. In some embodiments, the entire process, from acquisition of the lymphocytes from the donor to harvesting of the transfected lymphocytes, is completed within 4 hours.

In some embodiments, the entire process, from acquisition of the lymphocytes from the donor to implantation or cryopreservation of the transfected lymphocytes, is completed within 4 days. In some embodiments, the entire process, from acquisition of the lymphocytes from the donor to implantation or cryopreservation of the transfected lymphocytes, is completed within 3 days. In some embodiments, the entire process, from acquisition of the lymphocytes from the donor to implantation or cryopreservation of the transfected lymphocytes, is completed within 2 days. In some embodiments, the entire process, from acquisition of the lymphocytes from the donor to implantation or cryopreservation of the transfected lymphocytes, is completed within 36 hours. In some embodiments, the entire process, from acquisition of the lymphocytes from the donor to implantation or cryopreservation of the transfected lymphocytes, is completed within 24 hours. In some embodiments, the entire process, from acquisition of the lymphocytes from the donor to implantation or cryopreservation of the transfected lymphocytes, is completed within 18 hours.

As observed in the experimental examples, this improved autologous process does not require lymphocyte activation, T-cell enrichment or expansion.

“Lymphocyte activation,” or “lymphocyte stimulation,” refers to a process to stimulate a population of lymphocytes with one or more stimulating agents to produce a population of activated lymphocytes. Any combination of one or more suitable lymphocyte stimulating agents may be used to produce a population of activated lymphocyte including, but not limited to, an antibody or functional fragment thereof which targets a T-cell stimulatory or co-stimulatory molecule (e.g., anti-CD2 antibody, anti-CD3 antibody, anti-CD28 antibody, or functional fragments thereof) a T cell cytokine (e.g., any isolated, wildtype, or recombinant cytokines such as: interleukin 1 (IL-1), interleukin 2, (IL-2), interleukin 4 (IL-4), interleukin (IL-5), interleukin 6 (IL-6), interleukin 7 (IL-7), interleukin 12 (IL-12), interleukin 15 (IL-interleukin 21 (IL-21), interleukin 23 (IL-23), tumor necrosis factor α (TNFα)), or any other suitable mitogen (e.g., tetradecanoyl phorbol acetate (TPA), phytohaemagglutinin (PHA), concanavalin A (conA), lipopolysaccharide (LPS), pokeweed mitogen (PWM)) or natural ligand to a T-cell stimulatory or co-stimulatory molecule.

In some embodiments, lymphocyte activation employs an anti-CD3 antibody (or functional fragment thereof), an anti-CD28 antibody (or functional fragment thereof), or a combination of anti-CD3 and anti-CD28 antibodies in stimulating the population of lymphocytes.

Accordingly, in one embodiment, in the disclosed process, the lymphocytes are not made in contact with any exogenous agent (such as those exemplified above) that activates the lymphocytes.

“Lymphocyte enrichment” or “lymphocyte selection” refers to a process in which a subset of lymphocytes is selected for in a cell mixture. In certain aspects, CD3+ T cells may be selected from a cell mixture. In certain aspects, CD4+ and CD8+ T cells may be selected from a cell mixture. The selection process may be performed with antibodies (e.g., anti-CD3+ antibodies, anti-CD4+ antibodies, anti-CD8+ antibodies) or any other means for selection of cell sub-types known in the art.

“Lymphocyte expansion” refers to a process in which the lymphocytes are incubated at a suitable temperature (e.g., 37° C.) with sufficient supply to nutrients and other conditions (e.g., CO₂) to allow active splitting and proliferation. In some embodiments, the process of the instant disclosure does not include lymphocyte expansion. In some embodiments, the process of the instant disclosure includes an abbreviated lymphocyte expansion, which is not longer than 1 hour, 2 hours, 4 hours, 6 hours, 12 hours, 16 hours, 24 hour, 36 hours or 48 hours.

Preferably, in some embodiment, each step is performed in a closed system. Preferably, each step is performed in a culture medium free from human or animal serum.

The transfected lymphocytes generated from the instant processes can be suitably used for treating diseases. In some embodiments, the harvested lymphocytes, such as T cells, include a sufficient percentage of naïve T cells.

In some embodiments, at least 10%, 20%, 30%, 40%, 50%, 60%, 70%, 80% or 90% of the harvested lymphocytes are transfected lymphocytes. In some embodiments, at least 10%, 20%, 30%, 40%, 50%, 60%, 70%, 80% or 90% of the harvested lymphocytes have the transgene integrated to their genomes.

In some embodiments, at least 10%, 20%, 30%, 40%, 50%, 60%, 70%, 80% or 90% of the harvested T cells are transfected T cells. In some embodiments, at least 10%, 20%, 30%, 40%, 50%, 60%, 70%, 80% or 90% of the harvested T cells have the transgene integrated to the T cells' genomes.

In some embodiments, at least 10%, 20%, 30%, 40%, 50%, 60%, 70%, 80% or 90% of the harvested NK cells are transfected NK cells. In some embodiments, at least 10%, 20%, 30%, 40%, 50%, 60%, 70%, 80% or 90% of the harvested NK cells have the transgene integrated to the NK cells' genomes.

Various embodiments of the presently disclosed cell manufacturing process can be scaled up, as demonstrated in Example 8. In some embodiments, the scaled up process uses a sample for the transfection, which sample includes at least 10×10⁶ cells. In some embodiments, the sample includes at least 15×10⁶ cells, 20×10⁶ cells, 25×10⁶ cells, 30×10⁶ cells, 35×10⁶ cells, 6 cells, 45×10⁶ cells, 50×10⁶ cells, 60×10⁶ cells, 70×10⁶ cells, 80×10⁶ cells, 90×10⁶ cells, 100×10⁶ cells, 110×10⁶ cells, 120×10⁶ cells, 130×10⁶ cells, 140×10⁶ cells, 150×10⁶ cells, 200×10⁶ cells, 250×10⁶ cells, 300×10⁶ cells, 400×10⁶ cells, 500×10⁶, 600×10⁶ cells, 700×10⁶ cells, 800×10⁶ cells, 900×10⁶ cells, 1000×10⁶ cells, 1500×10⁶ or 2000×10⁶ cells.

In some embodiments, the transfection (e.g., electroporation) in the large scale process is carried out with the sample having a volume of at least 0.01 mL, or at least 0.05 mL, 0.1 mL, 0.2 mL, 0.3 mL, 0.4 mL, 0.5 mL, 0.6 mL, 0.7 mL, 0.8 mL, 0.9 mL, 1 mL, 1.1 mL, 1.2 mL, 1.3 mL, 1.4 mL, 1.5 mL, 2 mL, 3 mL, 4 mL, 5 mL, 6 mL, 7 mL, 8 mL, 9 mL, 10 mL, 12 mL, 15 mL, 18 mL, 20 mL, 22 mL, 25 mL, 30 mL, 40 mL, 50 mL, 60 mL, 70 mL, 80 mL, 90 mL, or 100 mL. In some embodiments, the transfection (e.g., electroporation) in the large scale process is carried out with the sample having a volume of not larger than 200 mL, 150 mL, 100 mL, 90 mL, 80 mL, 70 mL, 60 mL, 50 mL, 40 mL, 30 mL, 25 mL, 20 mL, 15 mL, 12 mL, 10 mL, 9 mL, 8 mL, 7 mL, 6 mL, 5 mL, 4 mL, 3 mL, 2 mL, 1.9 mL, 1.8 mL, 1.7 mL, 1.6 mL, 1.5 mL, 1.4 mL, 1.3 mL, 1.2 mL, 1.1 mL, 1 mL, 0.9 mL, 0.8 mL, 0.7 mL, 0.6 mL or 0.5 mL.

In some embodiments, in the larger scale process, at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, or 65% of lymphocytes in the harvested sample are naïve cells.

In some embodiments, in the larger scale process, at least 10%, 20%, 30%, 40%, 50%, 60%, 70%, 80% or 90% of the harvested lymphocytes are transfected lymphocytes. In some embodiments, at least 10%, 20%, 30%, 40%, 50%, 60%, 70%, 80% or 90% of the harvested lymphocytes have the transgene integrated to their genomes.

In any of the embodiments of the cell preparation process, the harvested cells can be subjected cell activation (if not activated prior to the transfection), selection and/or expansion. In some embodiments, the expansion can be carried out for 1, 2, 3, 4, 5, 6, 7, 8, 9, or 10 days.

### Compositions and Treatments

The cells, e.g., allogeneic cells, of the present disclosure can be used for treating various diseases and conditions, in particular cancer. In one embodiment, the cancer may comprise Wilms' tumor, Ewing sarcoma, a neuroendocrine tumor, a glioblastoma, a neuroblastoma, a melanoma, skin cancer, breast cancer, colon cancer, rectal cancer, prostate cancer, liver cancer, renal cancer, pancreatic cancer, lung cancer, biliary cancer, cervical cancer, endometrial cancer, esophageal cancer, gastric cancer, head and neck cancer, medullary thyroid carcinoma, ovarian cancer, glioma, lymphoma, leukemia, myeloma, acute lymphoblastic leukemia, acute myelogenous leukemia, chronic lymphocytic leukemia, chronic myelogenous leukemia, Hodgkin's lymphoma, non-Hodgkin's lymphoma, and urinary bladder cancer.

Another embodiment described herein is a method of treating a cancer in a subject in need thereof comprising administering an effective amount, e.g., therapeutically effective amount of a composition comprising a transfected cell of the present disclosure. Also provided are such compositions that include transfected lymphocytes disclosed herein and pharmaceutically acceptable excipients.

The quantity and frequency of administration will be determined by such factors as the condition of the patient, and the type and severity of the patient's disease, although appropriate dosages may be determined by clinical trials. In some embodiments, the cancer is characterized with the expression of an antigen targeted by the CAR or TCR molecule, such as CD19 and/or CD20.

In other embodiments, methods comprising administering a therapeutically effective amount of modified T cells contemplated herein or a composition comprising the same, to a patient in need thereof, alone or in combination with one or more therapeutic agents, are provided. In certain embodiments, the cells of the disclosure are used in the treatment of patients at risk for developing a cancer. Thus, the present disclosure provides methods for the treatment or prevention of a cancer comprising administering to a subject in need thereof, a therapeutically effective amount of the modified T cells of the disclosure.

One of ordinary skill in the art would recognize that multiple administrations of the compositions of the disclosure may be required to affect the desired therapy. For example a composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

In one embodiment, a subject in need thereof is administered an effective amount of a composition to increase a cellular immune response to a cancer in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions of the present disclosure, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

The methods for administering the cell compositions described herein includes any method which is effective to result in reintroduction of ex vivo genetically modified immune effector cells that either directly express an TCR or CAR in the subject or on reintroduction of the genetically modified progenitors of immune effector cells that on introduction into a subject differentiate into mature immune effector cells that express the TCR or CAR. One method comprises transfecting peripheral blood T cells ex vivo with a nucleic acid construct in accordance with the present disclosure and returning the transfected cells into the subject.

Although the foregoing disclosure has been described in some detail by way of illustration and example for purposes of clarity of understanding, it will be readily apparent to one of ordinary skill in the art in light of the teachings of this disclosure that certain changes and modifications may be made thereto without departing from the spirit or scope of the appended claims. The following examples are provided by way of illustration only and not by way of limitation. Those skilled in the art will readily recognize a variety of noncritical parameters that could be changed or modified to yield similar results.

All publications, patents, and patent applications mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present disclosure. To the extent that any of the definitions or terms provided in the references incorporated by reference differ from the terms and discussion provided herein, the present terms and definitions control. The contents of all references cited throughout this application are expressly incorporated herein by reference.

### EXAMPLES

### Example 1. Transposon-Mediated Bi-cistronic CAR Transfection and Integration

This example tested a transposon system introducing an anti-CD19 and anti-CD20 bi-cistronic CAR construct into host cells.

The bi-cistronic CAR, referred to herein as KITE-001, is expressed as a single-chain precursor which includes, from the N-terminus to the C-terminus, first signal peptide-anti-CD19 scFv-CD28 hinge and transmembrane domain-CD28 costimulatory domain-CD3ζ-T2A-second signal peptide-anti-CD20 scFv-CD8 hinge and transmembrane domain-4-1BB costimulatory domain-CD3ζ. Self-cleavage at the T2A results in the production of two separate CAR molecules.

The coding sequence of KITE-001, between the anti-CD19 portion and the anti-CD20 portion, share homology regions at the CD3t domains. Such homology was suspected to cause recombination and cryptic splicing. Accordingly, codon optimization and sequence wobbling were conducted to reduce nucleotide homology to be shorter than 9 nucleotides.

In a pilot study with a lentiviral vector, it was observed that expressed protein products included a number of variants. In particular, Variant 1 (V1), which accounted for about 1.5%-2% of all protein products, included the anti-CD19 only; Variant 14 (V14), which accounted for about 0.02-0.05% of all protein products, included a frameshift mutation in the anti-CD19 unit and thus was non-functional; Variant 15 (V15), which accounted for about 0.02-0.05% of all protein products, included a frameshift mutation in the heavy chain of the anti-CD20 scFv and thus only had anti-CD19 activity; and Variant 22 (V22), which accounted for about 0.02-0.05% of all protein products, had a deletion between anti-CD20 scFv and the CD8 hinge and thus is only functional toward CD19.

This example then employed a transposon system with a corresponding transposase for transfecting the KITE-001 coding sequence through electroporation (EP) into T cells. The transposon system included the coding sequence flanked by two sets of repeat sequences (CAGTTGAAGTCGGAAGTTTACATACACYTAAG, SEQ ID NO: 28, and YCCAGTGGGTCAGAAGTTTACATACACTMART, SEQ ID NO: 29). An example transposase sequence is

(SEQ ID NO: 30)
MGKSKEISQDLRKRIVDLHKSGSSLGAISKRLAVPRSSVQTIVRKYK
HHGTTQPSYRSGRRRVLSPRDERTLVRKVQINPRTTAKDLVKMLEET
GTKVSISTVKRVLYRHNLKGHSARKKPLLQNRHKKARLRFATAHGDK
DRTFWRNVLWSDETKIELFGHNDHRYVWRKKGEACKPKNTIPTVKHG
GGSIMLWGCFAAGGTGALHKIDGIMDAVQYVDILKQHLKTSVRKLKL
GRKWVFQHDNDPKHTSKVVAKWLKDNKVKVLEWPSQSPDLNPIENLW
AELKKRVRARRPTNLTQLHQLCQEEWAKIHPNYCGKLVEGYPKRLTQ
VKQFKGNATKY.

The transfected cells were then cultured and expanded. Unlike with the lentiviral system, variants V1 and V15 were entirely undetectable from the transfected cells, and no other variants were found to have greater than 0.02% prevalence. By contrast, variant V22 was detected at a noticeably higher level in the transposon based samples (Table 1).

TABLE 1
Percentages of Variants

| Variant | Lentiviral | Transposon |
|---|---|---|
| Not Translated | 0.13% | 0.0048% |
| V1 | 0.40% | 0% |
| V14 | 0.026% | 0.015% |
| V15 | 0.048% | 0% |
| V22 | 0.026% | 0.15% |

The stability of the transposon-based integration was monitored for three weeks. As shown in Tables 2 and 3, both LVV-generated and non-viral transposed CARs show in vitro stability of CD19/CD20 expression.

TABLE 2
Total CAR Expression (LVV)

| Days of | Donor 1 | Donor 1 | Donor 2 | Donor 2 |
|---|---|---|---|---|
| Manufacturing | NTD | Dual CAR | NTD | Dual CAR |
| Day 1 | | | | |
| Day 3 | 0.12 | 52.5 | 0.025 | 50.7 |
| Day 4 | 5 | 70 | 3.79 | 65.5 |
| Day 5 | | | | |
| Day 6 | | | | |
| Day 7 | | | | |
| Day 8 | 0.18 | 64.9 | 0.3 | 60.9 |
| Day 11 | 1.01 | 50.1 | 2.24 | 48.2 |
| Day 15 | 2.42 | 54.9 | 3.79 | 50.8 |
| Day 18 | 0.2 | 50.8 | 1.6 | 47.4 |
| Day 22 | 0.02 | 45 | 0.97 | 42.8 |

TABLE 3
Total CAR Expression (non-viral)

| Days post- | Donor 1 | Donor 1 | Donor 2 | Donor 2 |
|---|---|---|---|---|
| electroporation | (no EP) | Dual CAR | (no EP) | Dual CAR |
| Day 1 | 0.29 | 0.77 | 0.94 | 0.58 |
| Day 3 | | | | |
| Day 4 | 3.55 | 32.4 | 2.89 | 27.6 |
| Day 5 | | | | |
| Day 6 | | | | |
| Day 7 | | | | |
| Day 8 | 0.33 | 32.4 | 0.41 | 32.5 |
| Day 11 | 0.41 | 26.5 | 1.05 | 20 |
| Day 15 | 1.1 | 34.1 | 3.64 | 21.4 |
| Day 18 | 0.2 | 37.5 | 1.3 | 22.1 |
| Day 22 | 0.2 | 34.3 | 0.4 | 20.6 |

### Example 2. Single CAR Transfection Efficiency

The transfection efficiency of the transposon systems was then compared to that with the lentiviral vectors, with T cells from four different donors. In terms of percentage of transfected cells of total viable CD3+ cells (Table 4) and overall T cell viability (Table 5), both systems achieved excellent results. This result, therefore, demonstrates that, unlike the viral vector-based systems, the instant transposon system did not require T cell activation to achieve high transfection efficiency.

TABLE 4
Percentage of CAR+ of Viable CD3+ Cells

| Transposon | Control (no EP) | Research EP Scale | Scale-up EP Scale |
|---|---|---|---|
| Day 1 | 0.81 ± 0.32 | 36.20 ± 19.95 | 47.66 ± 11.35 |
| Day 4 | 0.2 ± 0.099 | 58.55 ± 14.56 | 73.90 ± 9.48 |
| Day 7 | 0.16 ± 0.095 | 38.26 ± 3.87 | 54.95 ± 13.28 |

| Lentiviral | NTD | LVV-TD |
|---|---|---|
| Day 0 | 0 ± 0 | 0 ± 0 |
| Day 3 | 0.050 ± 0.038 | 61.73 ± 4.07 |
| Day 7 | 0.020 ± 0.0052 | 77.83 ± 2.99 |

TABLE 5
T Cell Viability

| Transposon | Control (no EP) | Research EP Scale | Scale-up EP Scale |
|---|---|---|---|
| Day 0 | 94.55 ± 1.92 | 88.45 ± 2.27 | 93.78 ± 1.67 |
| Day 1 | 92.23 ± 1.48 | 73.8 ± 2.81 | 79.45 ± 3.10 |
| Day 4 | 94.65 ± 1.82 | 81.25 ± 4.34 | 85.63 ± 1.63 |
| Day 7 | 92.53 ± 0.90 | 86.9 ± 4.29 | 89.3 ± 1.01 |

| Lentiviral | NTD | LVV-TD |
|---|---|---|
| Day 0 | 85.25 ± 2.49 | 84.00 ± 3.39 |
| Day 1 | 62.13 ± 10.89 | 65.5 ± 12.03 |
| Day 3 | 82.38 ± 2.63 | 81.00 ± 4.06 |
| Day 4 | 86.00 ± 6.04 | 86.25 ± 3.34 |
| Day 5 | 87.25 ± 3.70 | 91.5 ± 1.66 |
| Day 6 | 92.00 ± 5.062 | 92.75 ± 1.09 |
| Day 7 | 92.25 ± 3.11 | 92 ± 2.24 |

### Example 3. Transposon-Mediated Reporter Gene or Single CAR Transfection and Integration

This example tested a transposon system introducing into host cells one of a GFP reporter gene, an anti-CD19 CAR construct with a 4-1BB costimulatory region or an anti-CD19 CAR construct with a CD28 costimulatory region.

As shown in Table 6, the expression of the integrated GFP reporter gene was maintained for over 21 days. By way of comparison, the non-integrated expression controls (no electroporation, minicircle, or plasmid only without transposase) dropped to below 1% within 7-10 days. This data, therefore, demonstrate that the transposase-mediated gene integration was stable. It is worth noting that, when the transposon was enclosed in a minicircle plasmid for delivery, the transfection efficiency was further improved, at all timepoints.

TABLE 6
Percentage of Cells Expressing GFP of Total Live CD3+ T Cells

| | | | | | minicircle | plasmid |
|---|---|---|---|---|---|---|
| Post- | minicircle 3 | minicircle 6.5 | plasmid 6.5 | | control | control |
| EP | μg + Transposase | μg + Transposase | μg + Transposase | no EP | 6.5 μg | 6.5 μg |
| Day 1 | 31.3 | 37.6 | 23.5 | 0.056 | 21 | 14.9 |
| Day 4 | 62 | 75.6 | 38.8 | 0.54 | 57.8 | 6.82 |
| Day 6 | 60.3 | 74.5 | 43.5 | 0.053 | 26.1 | 0.68 |
| Day 8 | 58.2 | 65.1 | 45.6 | 0.011 | 0.74 | 0.088 |
| Day 11 | 53.5 | 61.9 | 40.4 | 0.0053 | 0.49 | 0.15 |
| Day 14 | 49.75 | 56.2 | 35.4 | | 0.095 | 0.05 |
| Day 18 | 48.95 | 55 | 34.9 | | 0.075 | 0.07 |
| Day 21 | 50.3 | 56.4 | 35.8 | | 0.1 | 0.072 |

As shown in Table 7, the expression of the integrated anti-CD19 CAR construct with a 4-1BB costimulatory region was maintained for over 21 days. By way of comparison, the non-integrated expression control, plasmid only without transposase, dropped to below 1% within 7-10 days. This data, therefore, demonstrate that the transposon-mediated gene integration for this CAR construct was stable.

TABLE 7
Percentage of CD3 T Cells Expressing
anti-CD19 CAR with 4-1BB Costim

| | | Transposed |
|---|---|---|
| | | anti-CD19 CAR |
| Post-EP | Plasmid only | with 4-1BB Costim |
| Day 1 | 3.78 | 5.83 |
| Day 4 | 0.6 | 22.5 |
| Day 6 | 0.16 | 30.2 |
| Day 8 | | |
| Day 11 | 0.1 | 31.2 |
| Day 14 | 0.038 | 26.7 |
| Day 18 | 0.042 | 24.2 |
| Day 21 | 0.022 | 29.4 |

As shown in Table 8, the expression of the integrated anti-CD19 CAR construct with a CD28 costimulatory region was maintained for over 21 days. By way of comparison, the non-integrated expression control, no electroporation or plasmid only, dropped to below 1% within 7-10 days. This data, therefore, demonstrate that the transposon-mediated gene integration for this CAR construct was stable.

TABLE 8
Percentage of CD3 T Cells Expressing
anti-CD19 CAR with CD28 Costim

| | | | Transposed |
|---|---|---|---|
| | | | anti-CD19 CAR with |
| Post-EP | Control (no EP) | Plasmid only | CD28 Costim |
| Day 1 | 0.00589 | 11.7 | 28.2 |
| Day 3 | | | |
| Day 4 | 0 | 0.017 | 54.1 |
| Day 5 | | | |
| Day 6 | | | |
| Day 7 | | | |
| Day 8 | 0.00309 | 0.03 | 21.8 |
| Day 11 | 0 | 0.028 | 14 |
| Day 15 | 0.36 | 0.56 | 10.2 |
| Day 18 | 0.2 | 0.4 | 10 |
| Day 22 | 0.2 | 0.11 | 12 |

### Example 4. Development of Transposon-Mediated Autologous Process

The conventional viral vector-based autologous process takes 7 days or even weeks, from cell acquisition to implantation. This example attempted to develop an autologous process that can be completed as quickly as within a single day.

Following apheresis, T cells were enriched from the collected peripheral blood mononuclear cells (PMBC), which were then subjected to electroporation with the transposon construct (as tested in Example 1). T cells were harvested at 24 hours, or further activated and expanded.

The impacts of the duration of the process were assessed. As shown in Table 9,

TABLE 9
Basic Signature of Transposon-Mediated Autologous Processes

| | | | | CD45RA+ CCR7+ |
|---|---|---|---|---|
| | CAR (% over | Via- | CD4/ | CD62L+ CD27+ |
| | total live | bility | CD8 | CD28+ (% over |
| Day | CD3 T cells) | % | ratio | total live CD3 T cells) |
| 0 (before EP) | — | — | 5.51 | 52.5 |
| 1 (post EP) | 19.4 | 62 | 5.74 | 59.3 |
| 4 | 20.3 | 57 | 11.92 | 34.6 |
| 8 | 30.5 | 80 | 1.40 | 27.2 |

The processes (1 day, 3 days, and 7 days, at different cell counts) were tested with a Nalm6 mouse model of leukemia. The tumor burdens were measured following autologous cell implantation, and the results are presented in Table 10. A lentiviral vector-based autologous process (7 days) with the same CAR sequences was used as control.

TABLE 10
Tumor Burden Following T Cell Implantation (measured as BioLuminescence (photons/s))

| Days | | LVV CAR (7 days) | Transposed CAR (7 days) | Transposed CAR (1 day) |
|---|---|---|---|---|
| post-imp | Vehicle | 1E6 Cells | 1E6 Cells | 1E6 Cells |
| 5 | 4.6E+07 ± 4.1E−01 | 9.6E+07 ± 2.1E−01 | 5.4E+07 ± 4.0E−01 | 9.1E+07 ± 3.2E−01 |
| 8 | 7.5E+08 ± 3.3E−01 | 2.7E+08 ± 3.6E−01 | 7.5E+07 ± 3.6E−01 | 5.2E+08 ± 5.1E−01 |
| 13 | 1.4E+10 ± 1.3E−01 | 6.5E+05 ± 5.5E−02 | 9.3E+05 ± 4.7E−01 | 4.7E+06 ± 8.5E−01 |
| 16 | 3.2E+10 ± 1.4E−01 | 1.2E+06 ± 4.7E−02 | 6.3E+05 ± 7.5E−02 | 1.1E+06 ± 3.7E−02 |
| 20 | 4.2E+10 ± 3.2E−01 | 7.7E+05 ± 1.6E−01 | 7.0E+05 ± 1.0E−01 | 6.4E+05 ± 8.5E−02 |
| 22 | | 6.7E+05 ± 1.8E−01 | 6.6E+05 ± 1.5E−01 | 5.8E+05 ± 9.3E−02 |
| 26 | | 9.8E+05 ± 4.3E−01 | 1.0E+06 ± 6.7E−01 | 6.8E+05 ± 1.1E−01 |
| 29 | | 1.1E+06 ± 6.0E−01 | 3.6E+06 ± 1.6E+00 | 5.6E+05 ± 7.7E−02 |
| 34 | | 4.3E+06 ± 1.6E+00 | 3.6E+07 ± 1.9E+00 | 5.8E+05 ± 9.0E−02 |
| 37 | | 1.9E+07 ± 1.8E+00 | 1.1E+08 ± 1.9E+00 | 6.1E+05 ± 4.6E−02 |
| 41 | | 2.8E+07 ± 1.8E+00 | 2.1E+07 ± 1.9E+00 | 6.9E+05 ± 1.2E−01 |
| 44 | | 1.6E+06 ± 7.6E−01 | 7.7E+05 ± 1.4E−01 | 7.4E+05 ± 4.4E−02 |
| 48 | | 2.1E+06 ± 1.3E+00 | 8.2E+05 ± 1.4E−01 | 7.3E+05 ± 1.3E−01 |
| 54 | | 7.3E+06 ± 1.8E+00 | 8.5E+05 ± 1.4E−01 | 7.3E+05 ± 9.1E−02 |
| 58 | | 3.1E+07 ± 1.9E+00 | 8.1E+05 ± 2.3E−01 | 7.0E+05 ± 5.6E−02 |
| 62 | | 3.8E+08 ± 2.0E+00 | 1.1E+06 ± 7.3E−01 | 7.0E+05 ± 7.7E−02 |
| 65 | | 1.1E+09 ± 2.0E+00 | 2.0E+06 ± 1.2E+00 | 8.6E+05 ± 1.6E−01 |
| 68 | | 2.3E+09 ± 2.0E+00 | 1.8E+06 ± 9.5E−01 | 9.2E+05 ± 1.4E−01 |
| 75 | | 1.2E+07 ± 1.3E+00 | 4.6E+06 ± 1.6E+00 | 9.9E+05 ± 1.3E−01 |
| 82 | | 3.4E+08 ± 1.4E+00 | 1.6E+07 ± 1.8E+00 | 8.4E+05 ± 1.3E−01 |
| 89 | | 1.7E+08 ± 1.4E+00 | 1.2E+07 ± 1.8E+00 | 7.1E+05 ± 1.7E−01 |
| 5 | 6.1E+07 ± 2.6E−01 | 3.7E+07 ± 2.0E−01 | 4.5E+07 ± 3.6E−01 | 5.0E+07 ± 4.6E−01 |
| 8 | 9.0E+08 ± 2.6E−01 | 7.0E+08 ± 4.7E−01 | 6.6E+08 ± 4.9E−01 | 6.5E+08 ± 4.7E−01 |
| 13 | 1.7E+07 ± 5.3E−01 | 5.2E+09 ± 5.7E−01 | 5.8E+09 ± 3.5E−01 | 3.2E+09 ± 4.3E−01 |
| 16 | 7.5E+05 ± 3.6E−02 | 4.4E+08 ± 1.2E+00 | 7.4E+07 ± 9.3E−01 | 1.2E+08 ± 9.9E−01 |
| 20 | 7.9E+05 ± 5.9E−02 | 9.0E+05 ± 3.4E−02 | 6.7E+05 ± 1.2E−01 | 7.2E+05 ± 7.4E−02 |
| 22 | 4.9E+05 ± 6.9E−02 | 6.6E+05 ± 1.1E−01 | 6.1E+05 ± 9.4E−02 | 6.5E+05 ± 8.2E−02 |
| 26 | 6.7E+05 ± 1.5E−01 | 2.8E+06 ± 1.1E+00 | 6.4E+05 ± 1.4E−01 | 1.7E+06 ± 7.0E−01 |
| 29 | 6.5E+05 ± 8.7E−02 | 1.3E+06 ± 5.8E−01 | 7.1E+05 ± 1.9E−01 | 1.3E+06 ± 8.4E−01 |
| 34 | 6.3E+05 ± 7.7E−02 | 8.0E+05 ± 2.2E−01 | 5.8E+05 ± 1.7E−01 | 2.5E+06 ± 9.7E−01 |
| 37 | 5.7E+05 ± 9.0E−02 | 7.4E+05 ± 2.3E−01 | 5.6E+05 ± 8.9E−02 | 2.0E+07 ± 1.4E+00 |
| 41 | 6.7E+05 ± 2.0E−01 | 9.3E+05 ± 3.0E−01 | 7.3E+05 ± 1.8E−01 | 1.0E+07 ± 1.3E+00 |
| 44 | 9.9E+05 ± 7.2E−01 | 1.4E+06 ± 6.7E−01 | 6.2E+05 ± 1.2E−01 | 1.2E+08 ± 1.8E+00 |
| 48 | 8.0E+05 ± 2.7E−01 | 2.6E+06 ± 9.8E−01 | 7.4E+05 ± 5.7E−02 | 6.5E+08 ± 1.7E+00 |
| 54 | 2.8E+06 ± 1.5E+00 | 1.6E+07 ± 1.4E+00 | 6.9E+05 ± 1.3E−01 | 1.6E+10 ± 1.4E+00 |
| 58 | 1.2E+06 ± 1.2E+00 | 9.2E+07 ± 1.9E+00 | 5.6E+05 ± 1.0E−01 | 4.6E+09 ± 1.0E+00 |
| 62 | 1.1E+06 ± 9.0E−01 | 1.7E+09 ± 1.8E+00 | 6.6E+05 ± 6.6E−02 | 1.1E+10 ± 5.3E−01 |
| 65 | 1.0E+07 ± 1.8E+00 | 3.0E+09 ± 1.9E+00 | 7.2E+05 ± 1.6E−01 | 1.4E+10 ± 4.7E−01 |
| 68 | 4.3E+07 ± 1.9E+00 | 3.0E+09 ± 1.8E+00 | 7.2E+05 ± 5.9E−02 | 7.2E+09 ± 1.3E+00 |
| 75 | 8.3E+07 ± 1.9E+00 | 1.3E+08 ± 1.5E+00 | 9.3E+05 ± 9.8E−02 | 2.0E+10 ± 9.9E−01 |
| 82 | 2.2E+07 ± 1.6E+00 | 1.7E+08 ± 1.9E+00 | 8.2E+05 ± 1.2E−01 | |
| 89 | 4.3E+07 ± 1.7E+00 | 3.0E+08 ± 1.9E+00 | 9.1E+05 ± 1.6E−01 | |

The 7-day lentiviral vector-based process (10⁶ cells) led to significant reduction of tumor growth. The tumor burden peaked gradually at day 68 (about 2.3×10 9 photons/s), and then reduced to about 1.7×10 8 photons/s at day 89.

Among all the transposon-based processes, however, only the 7-day process at low dose (2×10⁵ cells) underperformed the lentiviral vector-based process at high dose (10⁶ cells). In particular, the 1-day process at high dose (10⁶ cells) and the 3-day process at low dose (2×10 5 cells) achieved tumor elimination at about day 20 (<10⁶ photons/s), and the tumor did not return at all. The high dose for the 3-day and 7-day processed performed excellently as well, while the low doses (2×10⁵ cells) for the 1-day process was comparable to the lentiviral vector-based process at high dose.

These results, therefore, demonstrate that the newly developed 1-day process for transposon-based autologous CAR-T achieved results far superior to those of the 7-day lentiviral vector-based process at the same dose (10⁶ cells). Even when used at a lower dose (2×10⁵ cells) than the 7-day lentiviral vector-based process (10⁶ cells), the results were still comparable. It is contemplated that such superior results from the newly developed, shortened process produced high quality CAR-T cells with higher naïve T cell populations favorable to cancer treatments.

### Example 5. Further Testing of the Autologous Process

This example further tested the shortened transposon-based autologous process with other CAR constructs. A construct targeting CD19 but including a 4-1BB costimulatory domain was used. Likewise, early (day 4) harvesting at both high and low doses greatly outperformed late (day 14) harvesting.

In each experiment, a fixed amount of plasmid DNA (gene cargo, 5 μg) and mRNA (encoding transposase, 5 μg) were used. The comparison is shown in Table 11.

TABLE 11
Transfection Efficiency for Each Construct

| Day post | | | | | |
|---|---|---|---|---|---|
| electro- | | | | | |
| poration | | | CD19 with | CD19 with | CD19/CD20 |
| (EP) | no EP | GFP | 41BB | CD28 | bicistronic |
| 1 | 0.00541 | 17.9 | 6.77 | 6.52 | 0.067 |
| 4 | 0.016 | 33.7 | 57.8 | 45.7 | 11.6 |
| 6 | 0.00442 | 30.5 | 37.1 | 30.2 | 12.3 |
| 8 | 0.071 | 28 | 33.4 | 20.8 | 10.7 |

Electroporation of plasmid DNA in general can result in 30-50% cell death at first 24 hr. Viability of cells produced by the new process were activated and expanded, and their viability were assessed as each stage. Table 12 shows the cell viability recover to 80-90% after 8 days, the same viability level as the no-electroporation cell group. Table 13 shows the viable cells grew well following the new manufacturing process with 30-40 fold expansion.

TABLE 12
T Cell Viability at Different Days

| Day post | | | | | |
|---|---|---|---|---|---|
| electro- | | | | | |
| poration | | | CD19 with | CD19 with | CD19/CD20 |
| (EP) | no EP | GFP | 41BB | CD28 | bicistronic |
| 1 | 86 | 56 | 54 | 51 | 57 |
| 4 | 77 | 67 | 56 | 65 | 63 |
| 6 | 85 | 90 | 85 | 88 | 87 |
| 8 | 90 | 91 | 88 | 90 | 87 |

TABLE 13
Fold Expansion at Different Days

| Day post | | | | | |
|---|---|---|---|---|---|
| electro- | | | | | |
| poration | | | CD19 with | CD19 with | CD19/CD20 |
| (EP) | no EP | GFP | 41BB | CD28 | bicistronic |
| 1 | 1 | 1 | 1 | 1 | 1 |
| 4 | 2.43 | 1.16 | 1.11 | 1.39 | 1.23 |
| 6 | 13.12 | 8.87 | 9.08 | 10.79 | 9.6 |
| 8 | 61.19 | 36.07 | 40.77 | 39.66 | 30.86 |

These data, therefore, demonstrate that the new non-viral process is applicable to various types of constructs. Moreover, cell viability can be recovered during subsequent expansion, demonstrating the excellent growth potential of the electroporated T cells.

### Example 6. Evaluation of Cells Produced from the New Process

This example evaluated the CAR-T cells produced from the shortened process, in particular with markers associated with naïve state of T cells.

CAR T cells were manufactured using the non-viral transposon approach described in Example 4, in static bags. The phenotyping data refers to percentage of T cells expressing CD45RA, CCR7, CD62L, CD27 and CD28 over total T cells (Table 14).

TABLE 14
Percentage of Naïve T Cells
(CD45RA⁺CCR7⁺CD62L⁺CD27⁺CD28⁺)

| Date | Starting material | Day 0 post transfection | Day 3 | Day 7 |
|---|---|---|---|---|
| % | 52.5 | 59.3 | 34.6 | 27.2 |

The earlier harvested cells had higher populations of juvenile populations, demonstrating the benefit of the expedited process.

The impact of T cell state, prior to transfection, on the transfection efficiency was also evaluated. Four types of T cells were examined, including CD45RA⁺CCR7⁺, CD45RA⁺CCR7⁻, CD45RA⁻CCR7⁺, and CD45RA⁻CCR7⁻. Cells are thawed with 2-hr recovery and then electroporated/transfection with DNA transposon and mRNA transposase. After 1 day-post transfection, cells are activated and expanded based on the new process. Harvested cells are labeled as Day 0, Day 3 and Day 7 based on activation day. As shown in Table 15, naïve T cells received more gene cargo than other subsets.

TABLE 15
Transfection efficiency in T cell subsets

| CAR % of | 1dpt (Day 0) | | | 4dpt (Day 3) | | | 8dpt (Day 7) | | |
|---|---|---|---|---|---|---|---|---|---|
| CD45RA + CCR7+ | 34.3 | 21.3 | 23.7 | 44.9 | 48.7 | 68.3 | 23.5 | 24.9 | 29.7 |
| CD45RA + CCR7− | 6.64 | 2.33 | 5.1 | 38.5 | 35.3 | 66 | 23.4 | 23.1 | 28.7 |
| CD45RA − CCR7+ | 8.74 | 5.92 | 11 | 35.3 | 42.5 | 67.5 | 17.5 | 20.7 | 27.8 |
| CD45RA − CCR7− | 3.88 | 3.4 | 4.95 | 29.3 | 29.1 | 59 | 18.2 | 18.3 | 19.4 |

### Example 7. Vector Copy Number (VCN) Measurement

This example developed a method to measure the copy number of constructs integrated into the genome of the target cell.

Primer sets were designed to target the construct (e.g., CD19 coding sequence), the plasmid backbone region (Amp), and a host reference gene (e.g., CDKN2A). Free DNA not integrated was digested with Dpn I. The VCN experiment was done with two separate ddPCR reactions—“CD19 vs host reference gene” and “AMP vs host reference gene.” CD19 VCN=2*CD19/host copies; Amp VCN=2*Amp/host copies (multiplied by the copy per genome of the reference (2 for diploid)). The integrated calculation was VCN=CD19 VCN−AMP VCN.

With this method, the average VCN in cells produced with the methods described above was determined to be about 5.07 (6.07 total DNA minus 1.0 free undigested DNA).

### Example 8. Comparison with TcBuster

This example compared the performance of the transposon system as tested in Example 1 (along with LVV) with TcBuster, another non-viral, transposon-based delivery system. All systems used a CD19 single CAR that included 4-1BB as the co-stimulatory domain. Transfected cells, that were frozen, were thawed overnight for CAR+ percentage measurement and in vitro cytotoxicity assays. The results are shown in Tables 16 and 17.

TABLE 16
Percentage of Transfected T Cells

| | Transposon only | TcBuster | Of Ex. 1 | LVV transduced |
|---|---|---|---|---|
| Donor #1 | 0.055 | 27 | 18.2 | 53.7 |
| Donor #2 | 0.54 | 20 | 12.9 | 52.7 |
| Donor #3 | 0.034 | 30.2 | 15.6 | 57.8 |

TABLE 17
Cytotoxicity

| | E:T | Plasmid only | | | TcBuster | | | Of Ex. 1 | | | Lentivirus | | |
|---|---|---|---|---|---|---|---|---|---|---|---|---|---|
| Donor | 1 to 1 | −32.1 | −23.6 | −27.4 | 97.2 | 97.5 | 98.3 | 98.5 | 98.9 | 98.8 | 98.5 | 98.6 | 98.5 |
| #1 | 1 to 3 | −41.5 | −36.2 | −33.6 | 71.7 | 76.3 | 64.6 | 72.3 | 75.4 | 74.6 | 77.5 | 76.3 | 75.1 |
| | 1 to 10 | −24.5 | −24.6 | −28.2 | 6.4 | 15.8 | 9.1 | 16 | 15.8 | 18.7 | 18.2 | 22.2 | 20.9 |
| Donor | 1 to 1 | −12.3 | −15.5 | −13.9 | 96.4 | 96.7 | 96.3 | 97.7 | 97.7 | 97.7 | 97.3 | 96.8 | 97.3 |
| #2 | 1 to 3 | −27.2 | −29.4 | −11.7 | 69.9 | 67.1 | 69.1 | 73.6 | 75.6 | 73.8 | 69.4 | 66.3 | 67.4 |
| | 1 to 10 | −17.2 | −14 | −7 | 7.6 | 12.2 | 7.7 | 5.4 | 18.3 | 25 | 12.7 | 5.4 | 10.3 |
| Donor | 1 to 1 | −19.5 | −19.5 | −28.4 | 98.8 | 98.8 | 98.8 | 99.3 | 99 | 99.1 | 99.1 | 98.5 | 99 |
| #3 | 1 to 3 | −33.6 | −36.3 | −25.8 | 87.8 | 87.3 | 86.7 | 93.6 | 91.7 | 92.2 | 74.2 | 74.5 | 69.7 |
| | 1 to 10 | −22.4 | −25.4 | −21.3 | 18.2 | 16.2 | 31.5 | 20.5 | 22.8 | 35.3 | 13.3 | 9.8 | 16.3 |

The Nalm6 animal model as used in Examples 4 and 5 was employed to test the tumor inhibition efficacy of these CAR cells. The results are shown in Table 18.

TABLE 18
Tumor Inhibition Efficacy

| | | G3. UT | | | |
|---|---|---|---|---|---|
| Day | G1. Vehicle | Transposon Only | G4. TcBuster | G5. Of Ex. 1 | G6. Lentivirus Td |
| 0 | 1.2E+06 ± 9.6E+04 | 1.3E+06 ± 1.1E+05 | 1.2E+06 ± 1.3E+05 | 1.2E+06 ± 1.6E+05 | 1.1E+06 ± 2.2E+05 |
| 3 | 2.4E+06 ± 3.5E+05 | 2.8E+06 ± 3.7E+05 | 2.6E+06 ± 4.4E+05 | 2.8E+06 ± 3.3E+05 | 1.8E+06 ± 2.2E+05 |
| 5 | 9.5E+06 ± 2.0E+06 | 9.3E+06 ± 2.0E+06 | 1.0E+07 ± 1.5E+06 | 9.2E+06 ± 1.3E+06 | 8.1E+06 ± 8.7E+05 |
| 10 | 9.5E+08 ± 2.7E+08 | 7.1E+08 ± 7.8E+07 | 1.2E+06 ± 1.3E+05 | 2.2E+06 ± 5.3E+05 | 2.1E+06 ± 5.9E+05 |
| 13 | 1.6E+10 ± 3.8E+09 | 1.0E+10 ± 2.0E+09 | 1.6E+06 ± 1.4E+05 | 2.2E+06 ± 4.1E+05 | 2.3E+06 ± 3.5E+05 |
| 17 | | | 1.6E+06 ± 1.7E+05 | 3.6E+06 ± 1.4E+06 | 2.6E+06 ± 1.5E+06 |
| 20 | | | 1.9E+06 ± 4.2E+05 | 1.3E+07 ± 1.3E+07 | 1.2E+07 ± 1.7E+07 |
| 25 | | | 1.0E+07 ± 8.3E+06 | 5.2E+07 ± 4.8E+07 | 4.8E+08 ± 8.9E+08 |
| 27 | | | 3.0E+07 ± 2.8E+07 | 1.2E+08 ± 1.2E+08 | 1.4E+09 ± 2.6E+09 |
| 31 | | | 2.3E+08 ± 2.5E+08 | 5.9E+08 ± 6.0E+08 | 3.2E+09 ± 5.8E+09 |
| 34 | | | 1.1E+09 ± 1.3E+09 | 2.4E+09 ± 2.5E+09 | 6.1E+09 ± 9.6E+09 |
| 38 | | | 8.9E+09 ± 1.1E+10 | 9.1E+09 ± 9.0E+09 | 7.3E+09 ± 4.8E+09 |

These data, therefore, demonstrate comparable in vitro cytotoxicity and in vivo efficacy of CAR T cells manufactured from either TcBuster, the transposon system of Example 1, or the LVV systems.

### Example 9. Scale Up Manufacturing Process

This example developed a large scale manufacturing process based on the small scale pilot study in Example 4.

In the small scale process, 5×10⁶ cells were used in electroporation, in a 100 μL volume. In the large scale process, 50×10⁶ to 100×10⁶ cells were used, in a 1 mL volume. In a comparative study, cells harvested from both small scale and large scale processes were subjected to activation (with anti-CD3 antibody) and expansion for up to 7 days.

In terms of cell number fold changes, there was no significant difference between the processes (p=08725). The large scale process, however, showed significantly higher viability than the small scale process using paired t test (p=0.0090).

Finally, the transfection efficacy was also evaluated for the large scale process as compared to the small scale one. The large scale process showed significantly higher transfection efficiency (p=0.0115) than the small scale process using paired t test.

### Example 10. Large Payload Transfection: Tricistronic

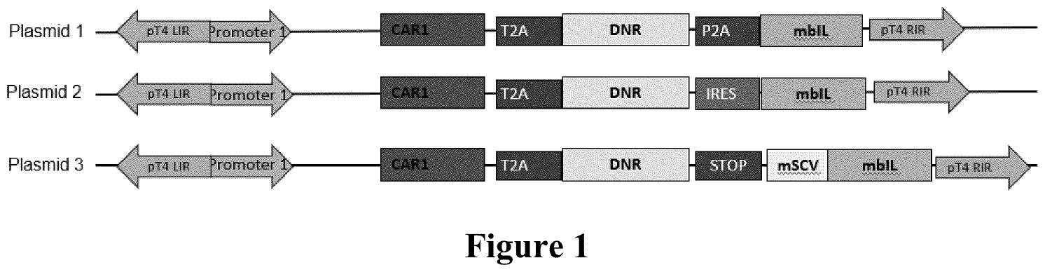

This example describes separate transfection of three different large payload plasmids utilizing a Sleeping Beauty transposon system, as depicted in FIG. 1. The three different plasmid constructs (Plasmid 1, Plasmid 2, and Plasmid 3) were electroporated into cells.

Plasmids 1-3 each individually encode a CAR, a dominant negative receptor (DNR), and a membrane bound interleukin receptor (mbIL). Plasmid 1 is a tri-cistronic construct which is 7725 bp in length including an insert of 4835 bp expressed as one mRNA transcript and a single long polypeptide which is cleaved post-translation at the T2A and P2A sites to provide a CAR, a DNR, and a mbIL. The Plasmid 2 is a tricistronic construct which is 8203 bp in length including a 5313 bp insert expressed as one mRNA transcript and two separate polypeptides. The first polypeptide includes the CAR and DNR which is cleaved post-translation at the T2A site. The second polypeptide is the mbIL. The Plasmid 3 is a tricistronic construct which is 8132 bp in length including an insert of 5242 bp expressed as two mRNA transcripts and two polypeptides. The first polypeptide includes the CAR and DNR which is cleaved post-translation at the T2A site. The second polypeptide is the mbIL.

TABLE 19
Transfection efficiency

| DPT\single | Plasmid 1 | | | Plasmid 2 | | | Plasmid 3 | | |
|---|---|---|---|---|---|---|---|---|---|
| TD% | CAR | DNR | mbIL | CAR | DNR | mbIL | CAR | DNR | mbIL |
| D1 | 2.3 | 1.1 | 1.6 | 1.7 | 1.7 | 0.3 | 1.4 | 1.3 | 0.3 |
| D4 | 23.3 | 15.9 | 17.3 | 30.2 | 26.8 | 4.6 | 14.5 | 13.1 | 4.3 |
| D6 | 25.4 | 5.8 | 15.5 | 24.4 | 20.4 | 4.9 | 10.9 | 8.6 | 3.1 |
| D8 | 15.2 | 4.8 | 9.1 | 16.5 | 12.1 | 3.3 | 8.4 | 5.6 | 1.5 |

### Example 11. Large Payload Transfection: Quad-Cistronic

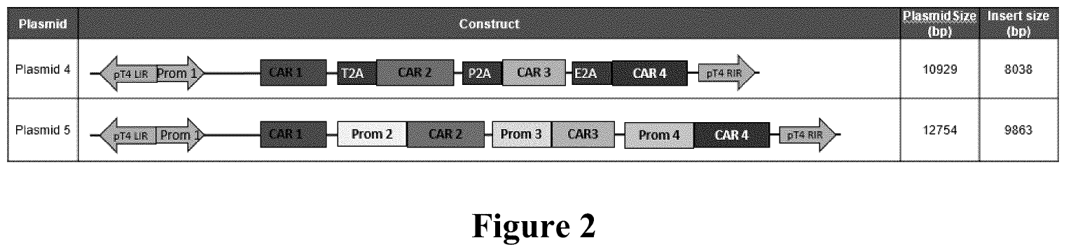

This example describes separate transfection of two different very large payload plasmids, as depicted in FIG. 2. The two different plasmid constructs (Plasmid 4 and Plasmid 5 were electroporated into cells with a single pulse. Table 20 shows CAR expression following transfection with Plasmid 4 having an 8 kb insert with transposase. Table 21 shows CAR expression following transfection with Plasmid 4 without adding transposase. Table 22 shows CAR expression following transfection with Plasmid 5 having a 10 kb insert with transposase. Table 23 shows CAR expression following transfection with Plasmid 5 without adding transposase.

TABLE 20
8 kb Insert (Plasmid 4)
Plasmid 4_Transposase

| DPT/Single | | | | | Overall |
|---|---|---|---|---|---|
| TD % | CAR1 | CAR2 | CAR3 | CAR4 | TG % |
| Day 1 | 6.54 | 12.4 | 16.6 | 13.5 | 18.81 |
| Day 4 | 21.6 | 31.6 | 19.6 | 34.6 | 35.47 |
| Day 6 | 22.2 | 27.6 | 24.9 | 29.1 | 29.83 |
| Day 8 | 21.6 | 24.4 | 26.4 | 31.2 | 31.72 |

TABLE 21
no transposase (Plasmid 4)
Plasmid 4 _Plasmid Only

| DPT/Single | | | | | Overall |
|---|---|---|---|---|---|
| TD % | CAR1 | CAR2 | CAR3 | CAR4 | TG % |
| Day 1 | 5.18 | 9.93 | 13.1 | 11 | 14.28 |
| Day 4 | 1.14 | 1.19 | 0.76 | 2.79 | 3.49 |
| Day 6 | 0.92 | 0.71 | 0.52 | 2.02 | 2.92 |
| Day 8 | 0.49 | 0.89 | 0.41 | 1.61 | 2.46 |

TABLE 22
10 kb Insert (Plasmid 5)
Plasmid 5_Transposase

| DPT/Single | | | | | Overall |
|---|---|---|---|---|---|
| TD % | CAR1 | CAR2 | CAR3 | CAR4 | TG % |
| Day 1 | 25.2 | 5.95 | 23.9 | 24.3 | 29.32 |
| Day 4 | 53.7 | 4.27 | 14.9 | 43.8 | 53.96 |
| Day 8 | 18 | 4.1 | 10 | 10.9 | 19.98 |

TABLE 23
no transposase (Plasmid 5)
Plasmid 5_Plasmid Only

| DPT/Single | | | | | Overall |
|---|---|---|---|---|---|
| TD % | CAR1 | CAR2 | CAR3 | CAR4 | TG % |
| Day 1 | 23.6 | 5.7 | 21.9 | 23 | 24.64 |
| Day 4 | 9.86 | 1.82 | 0.49 | 4.86 | 5.45 |
| Day 8 | 0.96 | 1.18 | 2.83 | 0.64 | 1.99 |

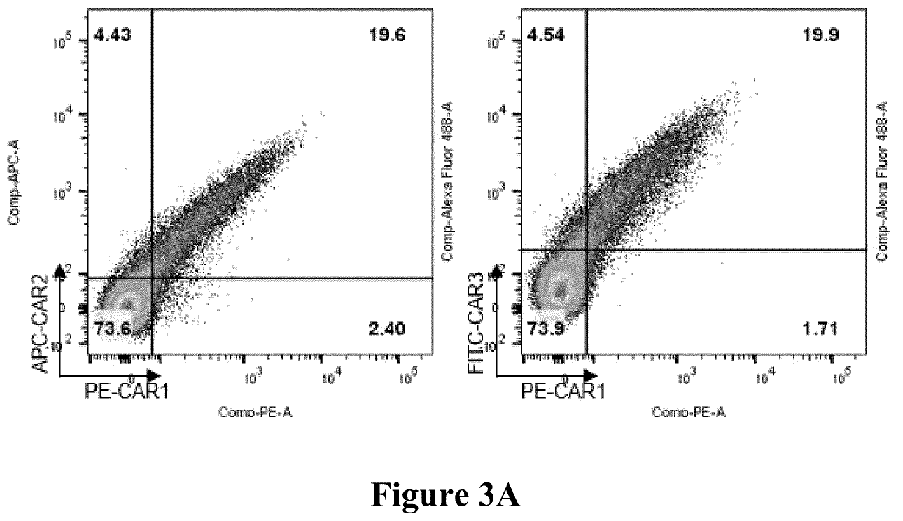

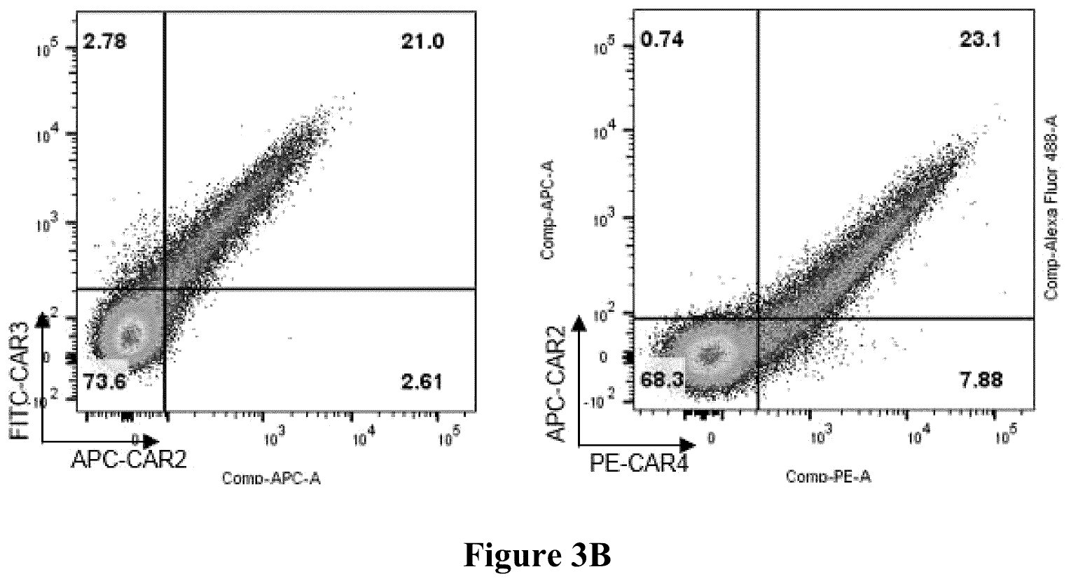

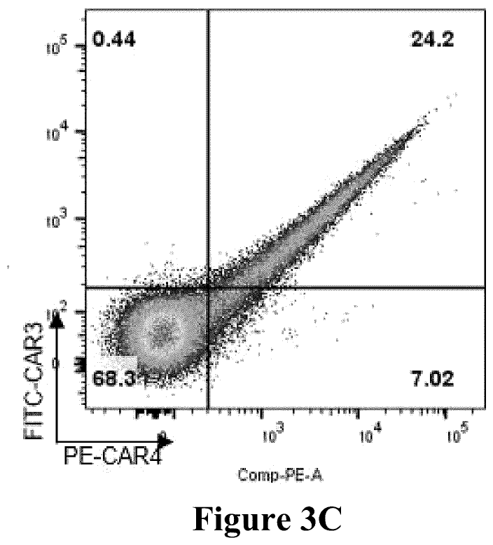

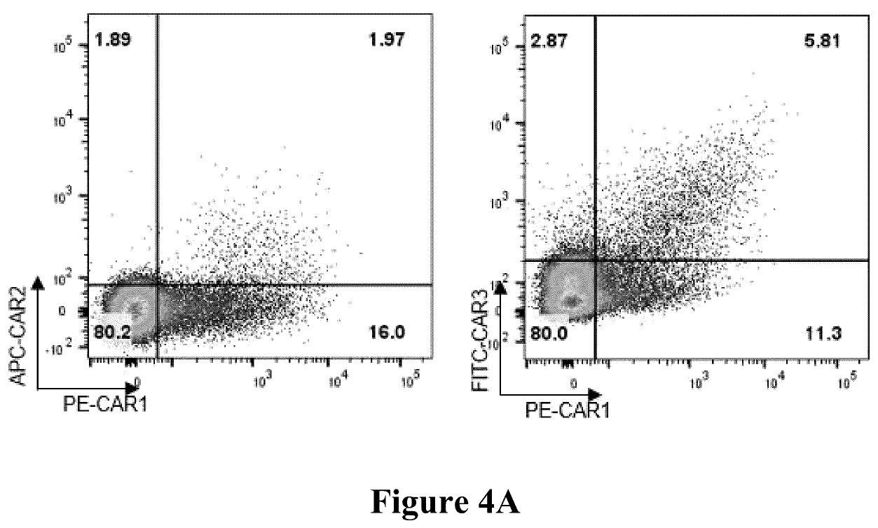

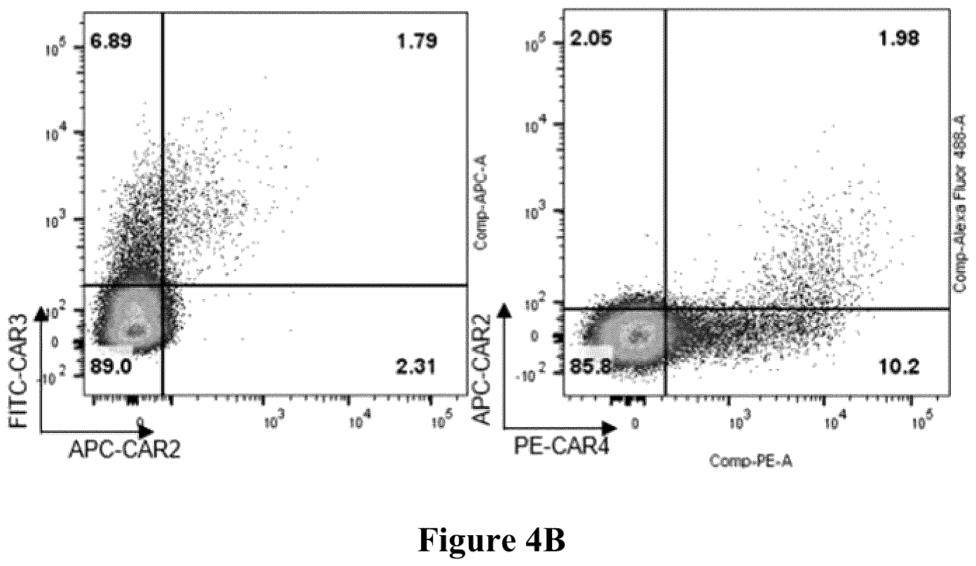

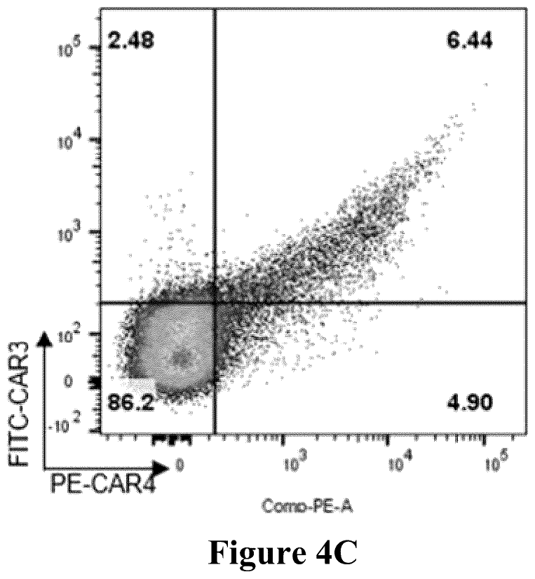

FIGS. 3A-3C show that the CAR expression at Day 8 post-electroporation for each of the four CARs of the quad-cistronic Plasmid 4 was relatively proportional. FIGS. 4A-4C show that the CAR expression at Day 8 post-electroporation for each of the four CARs of the quad-cistronic Plasmid 5 was also relatively proportional.

### Example 12. Stability and Re-stimulation

This example describes CAR expression following non-viral transfection of cells with the Plasmid 4. At Day 11 post-electroporation, transfected cells were restimulated with anti-CD3 antibody. Table 24 shows CAR expression without restimulation. Table 25 shows CAR expression with restimulation at Day 11. The data shows stability of transgene expression from the large payload construct, Plasmid 4.

TABLE 24
CAR Expression Without Restimulation
Plasmid 4_20ug DNA_DNA:mRNA = 1:16 No-Restim

| | | | | | | Overall TD% |
|---|---|---|---|---|---|---|
| | | | | | Overall | from Plasmid |
| | CAR1 | CAR2 | CAR3 | CAR4 | TD% | only |
| Day 1 | 6.54 | 12.4 | 16.6 | 13.5 | 18.81 | 14.28 |
| Day 4 | 21.6 | 31.6 | 19.6 | 34.6 | 35.47 | 3.49 |
| Day 6 | 22.2 | 27.6 | 24.9 | 29.1 | 29.83 | 2.92 |
| Day 8 | 21.6 | 24.4 | 26.4 | 31.2 | 31.72 | 2.46 |
| Day 11 | 23.1 | 24.5 | 29.5 | 38.9 | 39.9 | 3.06 |
| Day 15 | 16.8 | 21.4 | 18 | 35 | 35 | 1.66 |
| Day 19 | 9.34 | 13.7 | 11.5 | 25.1 | 17.03 | 2.12 |

TABLE 25
CAR Expression With Restimulation at Day 11
Plasmid 4_20ug DNA_DNA:mRNA = 1:16 Restim

| | | | | | | Overall TD% |
|---|---|---|---|---|---|---|
| | | | | | Overall | from Plasmid |
| | CAR1 | CAR2 | CAR3 | CAR4 | TD% | only |
| Day 1 | 6.54 | 12.4 | 16.6 | 13.5 | 18.81 | 14.28 |
| Day 4 | 21.6 | 31.6 | 19.6 | 34.6 | 35.47 | 3.49 |
| Day 6 | 22.2 | 27.6 | 24.9 | 29.1 | 29.83 | 2.92 |
| Day 8 | 21.6 | 24.4 | 26.4 | 31.2 | 31.72 | 2.46 |
| Day 11 | 23.1 | 24.5 | 29.5 | 38.9 | 39.9 | 3.06 |
| Day 12 | 37 | 40.1 | 45 | 56.1 | 54.52 | 2.24 |
| Day 15 | 22.9 | 40.4 | 35.1 | 49.5 | 51.25 | 2.74 |
| Day 19 | 13.6 | 16.3 | 10.2 | 28 | 19.51 | 3.94 |

### Example 13. Promoter Evaluation

This example describes the evaluation of different promoters in transposon encoding plasmids. The same gene of interest cassettes were designed under different promoters and tested in a non-viral cell culture process to evaluate gene expression kinetics driven by different promoters. The results show that the transposon transfection system works for different promoters, providing CAR expression from single CAR and dual CAR plasmids with varied promoters. Further, different promoters provide different levels of CAR expression providing for selection of CAR expression level based on selection of promotor.

TABLE 26
Plasmids for Promoter Evaluation

| | CAR | Promoter |
|---|---|---|
| Plasmid 6 | Single CAR | 1 |
| Plasmid 7 | Dual CAR | 2 |
| Plasmid 8 | Dual CAR | 1 |
| Plasmid 9 | Dual CAR | 3 |
| Plasmid 10 | Dual CAR | 4 |

TABLE 27
CAR Expression with Sleeping Beauty Transposon System
Plasmid + SB (Transposon + Transposase)

| | | | | | | | No |
|---|---|---|---|---|---|---|---|
| CAR 1 % | 6_SB | 7_SB | 8_SB | 9_SB | 10_SB | non EP | DNA |
| Day 1 | 27.4 | 3.13 | 9.68 | 2.12 | 2.09 | 0.04 | 0.08 |
| Day 4 | 33 | 7.23 | 28.9 | 16.6 | 7.27 | 0.08 | 0.13 |
| Day 6 | 17 | 6.25 | 12 | 13.6 | 9.81 | 0.3 | 0.42 |
| | | | | | | | No |
| CAR 2 % | | 7_SB | 8_SB | 9_SB | 10_SB | non EP | DNA |
| Day 1 | | 0.94 | 12 | 3.33 | 3.45 | 0.46 | 0.14 |
| Day 4 | | 4.9 | 28.6 | 14.2 | 4.92 | 0.22 | 0.26 |
| Day 6 | | 4.84 | 11.5 | 12.8 | 8.25 | 1.1 | 0.39 |
| Overall | | | | | | | No |
| TD % | | 7_SB | 8_SB | 9_SB | 10_SB | non EP | DNA |
| Day 1 | | 3.47 | 12.68 | 3.97 | 3.85 | 0.73 | 0.31 |
| Day 4 | | 7.73 | 31.55 | 17.19 | 7.54 | 0.30 | 0.35 |
| Day 6 | | 6.16 | 12.21 | 13.74 | 9.68 | 1.13 | 0.56 |

TABLE 28
CAR Expression without Sleeping Beauty Transposase
Plasmid Only (Transposon only)

| | | | | | | non | No |
|---|---|---|---|---|---|---|---|
| CAR 1 % | 6_only | 7_only | 8_only | 9_only | 10_only | EP | DNA |
| Day 1 | 19.9 | 2.24 | 12.4 | 1.88 | 1.6 | 0.04 | 0.08 |
| Day 4 | 2.91 | 0.49 | 3.61 | 3.62 | 0.55 | 0.08 | 0.13 |
| Day 6 | 0.34 | 0.35 | 0.37 | 0.38 | 0.24 | 0.3 | 0.42 |
| | | | | | | non | No |
| CAR 2 % | | 7_only | 8_only | 9_only | 10_only | EP | DNA |
| Day 1 | | 0.62 | 15.1 | 3.1 | 2.44 | 0.46 | 0.14 |
| Day 4 | | 0.16 | 0.86 | 1.85 | 0.12 | 0.22 | 0.26 |
| Day 6 | | 0.13 | 0.25 | 0.26 | 0.14 | 1.1 | 0.39 |
| Overall | | | | | | non | No |
| TD % | | 7_only | 8_only | 9_only | 10_only | EP | DNA |
| Day 1 | | 2.55 | 15.64 | 3.62 | 3.02 | 0.73 | 0.31 |
| Day 4 | | 0.63 | 3.78 | 3.83 | 0.63 | 0.30 | 0.35 |
| Day 6 | | 0.27 | 0.41 | 0.44 | 0.25 | 1.13 | 0.56 |

### Example 14. Testing of Transgene Expression Nanoplasmid vs. pCDL Plasmid

This example describes the evaluation of different backbone sizes comparing the expression of a gene for an anti-CD19 CAR after transfection by electroporation with an anti-CD19 CAR encoding transposon on a pCDL plasmid versus after transfection by electroporation with the same transposon on a nanoplasmid. The results of Table 29 show that higher expression levels were achieved for anti-CD19 CAR by transfection with the nanoplasmid. The ratio of transposon DNA to transposase mRNA was 1:4.

TABLE 29
anti-CD19 CAR Expression Nanoplasmid vs. pCDL plasmid

| | pCDL Plasmid | Nanoplasmid | Nanoplasmid | |
|---|---|---|---|---|
| Time | 8 ug | 5.6 ug | 8 ug | Non-EP |
| Day 1 | 11.8 | 12.8 | 22.5 | 0.43 |
| Day 4 | 53.9 | 68.9 | 77.9 | 2.07 |
| Day 6 | 39 | 53.3 | 65.2 | 1.55 |
| Day 8 | 30.8 | 44.9 | 52 | 0.81 |

While a number of embodiments have been described, it is apparent that the disclosure and examples may provide other embodiments that utilize or are encompassed by the compositions and methods described herein. Therefore, it will be appreciated that the scope of is to be defined by that which may be understood from the disclosure and the appended claims rather than by the embodiments that have been represented by way of example.

1. A transposon comprising a transgene encoding a polypeptide that comprises a first chimeric antigen receptor (CAR) and a second CAR, wherein the first CAR and the second CAR each comprises a single chain fragment (scFv), a transmembrane domain, and an immunoreceptor tyrosine-based activation motif (ITAM).

2. The transposon of claim 1, wherein the transposon is a DNA transposon selected from the group consisting of a Sleeping Beauty transposon, a piggyBac transposon, and a Tc Buster transposon, or a retro-transposon.

3. The transposon of claim 2, wherein the transposon is a Sleeping Beauty transposon or a Tc Buster transposon.

4. The transposon of claim 1, wherein the transgene is at least 5000 nucleotides in length.

5. The transposon of claim 4, wherein the transgene is at least 6000 nucleotides in length.

6. The transposon of claim 1, wherein the coding sequence for each ITAM in the transgene is codon-optimized to not have sequence identity to one another of 12 consecutive nucleotides or longer.

7. The transposon of claim 6, wherein the coding sequence for each ITAM in the transgene is codon-optimized to not have sequence identity to one another of 9 consecutive nucleotides or longer.

8. The transposon of claim 1, wherein the ITAM is a cytoplasmic signaling sequence derived from a protein selected from the group consisting of TCRzeta, FcRgamma, FcRbeta, CD3gamma, CD3delta, CD3epsilon, CD3ζ; CD5, CD22, CD79a, CD79b and CD66d.

9. The transposon of claim 8, wherein the ITAM is derived from CD3ζ, CD3epsilon or both.

10. The transposon of claim 1, wherein the first CAR and second CAR each further comprises an intracellular costimulatory domain.

11. The transposon of claim 10, wherein the intracellular costimulatory domain is a signaling region of a protein selected from the group consisting of DAP-10, CD28, OX-40, 4-1BB (CD137), CD2, CD7, CD27, CD30, CD40, programmed death-1 (PD-1), inducible T cell costimulator (ICOS), lymphocyte function-associated antigen-1 (LFA-1, CD11a/CD18), CD3 gamma, CD3 delta, CD3 epsilon, CD247, CD276 (B7-H3), tumor necrosis factor superfamily member 14, TNFSF14, LIGHT), NKG2C, Ig alpha (CD79a), Fc gamma receptor, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, CDS, GITR, BAFFR, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, IA4, CD49D, ITGA6, VLA-6, CD49f, ITGAD (CD11d), ITGAE (CD103), ITGAL (CD11a), ITGAM (CD11b), ITGAX (CD11c), ITGB1, CD29, ITGB2, CD18, ITGB7, NKG2D, TNFR2, TRANCE (RANKL), DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG (Cbp), CD19a, a ligand that specifically binds with CD83, and combinations thereof.

12. The transposon of claim 10, wherein the intracellular costimulatory domain is a signaling region of DAP-10, 4-1BB or CD28.

13. A cell comprising the transposon of claim 1.

14. The cell of claim 13, which is a T cell, NK cell, NKT cell, monocyte, macrophage, or a precursor cell thereof.

15. The cell of claim 13 or 11, further comprising a heterologous transposase.

16. The cell of claim 15, wherein the heterologous transposase is selected from the group consisting of a piggyBac® transposase, a piggy-Bac® like transposase, a Super piggyBac® (SPB) transposase, a piggyBac transposase, a Sleeping Beauty transposase, a hyperactive Sleeping Beauty (SB100X) transposase, Helitron transposase, a Tol2 transposase, a TcBuster transposase or a hyperactive TcBuster transposase.

17. The cell of claim 16, wherein the heterologous transposase is a Sleeping Beauty transposase SB100X or a piggyBac transposase.

18-51. (canceled)
